(19) United States
(12) Patent Application Publication
(10) Pub. No.: US 2006/0052351 A1
(43) Pub. Date: Mar. 9, 2006

(54) Oils enriched with diacylglycerols and phytosterol esters for use in the reduction of blood cholestrol and triglycerides and oxidative stress

(75) Inventors: Dorit Platt, Shimshit (IL); Dori Pelled, Hod Hasharon (IL); Avidor Shulman, Kiryat Tivon (IL)

Correspondence Address: FLEIT KAIN GIBBONS GUTMAN BONGINI & BIANCO, 21355 EAST DIXIE HIGHWAY, SUITE 115, MIAMI, FL, 33180, US

(73) Assignee: ENZYMOTEC LTD., MIGDAL HAEMEQ (IL)

(21) Appl. No.: 11/199,584

(22) Filed: Aug. 8, 2005

Related U.S. Application Data
(63) Continuation-in-part of application No. PCT/IL04/00131, filed on Feb. 10, 2004.

(30) Foreign Application Priority Data
Feb. 10, 2003 (IL) 154381
Mar. 27, 2003 (IL) 155136

(51) Int. Cl. A61K 31/56; A61K 31/225

(52) U.S. Cl. 514/171; 514/547

(57) ABSTRACT

Disclosed is the use of a composition comprising a combination of diacylglycerol(s) (DAG), mainly 1,3-diacylglycerol(s), and phytosterol and/or phytostanol ester(s) (PSE) dissolved or dispersed in edible oil and/or edible fat, particularly olive, canola and fish oil, in the manufacture of nutritional supplements and orally administrable pharmaceutical preparations which are capable of reducing blood levels of both cholesterol and triglycerides and/or for lowering serum, serum LDL and macrophage oxidation levels, inhibiting the formation of foam cells and/or preventing the deleterious effects generated by lipid-induced oxidative stress. In addition, the composition of the invention, as well as the pharmaceutical preparations thereof, are suitable for the treatment and prevention of conditions related to atherosclerosis, such as cardiovascular disease (CVD), coronary heart disease (CHD) and diabetes mellitus.

### CROSS REFERENCE TO RELATED APPLICATIONS

This application is a Continuation in Part of International Patent Application No. PCT/IL2004/000131 filed Feb. 10, 2004.

### FIELD OF THE INVENTION

The present invention relates to a combination of diacylglycerol(s) (DAG), mainly 1,3-diacylglycerol(s), and phytosterol and/or phytostanol ester(s) (PSE), optionally dissolved or dispersed in edible oil and/or edible fat, which may be used in the manufacture of nutritional supplements and orally administrable pharmaceutical preparations for reducing serum levels of both cholesterol and triglycerides. The combination also exhibits LDL-anti-oxidative properties, and is suitable for the treatment and prevention of cardiovascular disease (CVD) and coronary heart disease (CHD).

### BACKGROUND OF THE INVENTION

All publications mentioned throughout this application are fully incorporated herein by reference, including all references cited therein.

Coronary Artery Disease (like atherosclerosis) is the major cause of morbidity and mortality in the Western world and its pathogenesis involves complicated interactions between cells of the arterial wall, blood cells, and plasma lipoproteins [Ross R. (1993) Nature 362: 801-809; Glass C. K. and Witztum J. L. (2001) Cell 104:503-516]. Today, it is common knowledge that lowering cholesterol levels reduces the risk of heart attacks, strokes and other forms of atherosclerotic vascular disease. In addition, many recent studies have shown that oxidative stress is a mechanism with a central role in the pathogenesis of atherosclerosis, cancer, and other chronic diseases. In this scenario, a key role is played by macrophages in the sub-endothelial space, which are activated by oxidized low-density lipoproteins (ox-LDL). Recently, endothelial dysfunction due to oxidative stress was identified as a priming factor in the course of the development of atherosclerotic plaques.

The early atherosclerotic lesion is characterized by foam cells derived from cholesterol loaded macrophages [Gerrity R. G. (1981) Am. J. Pathol. 103:181-190; Schaffner T. et al. (1980) Am. J. Pathol. 100:57-80]. Macrophage cholesterol accumulation and foam cell formation are the hallmark of early atherogenesis and most of the cholesterol in these cells is derived from plasma low-density lipoprotein (LDL). Native LDL however, has to undergo some modifications in order to cause extensive macrophage cholesterol accumulation [Brown M. S. and Goldstein J. L. (1983) Annu. Rev. Biochem. 52:223-261; Kaplan M. and Aviram M. (1999) Clin. Chem. Lab. Med. 37:777-787; Aviram M. (1993) Atherosclerosis 98:1-9.; Steinberg D. et al. (1989) N. Engl. J. Med. 320: 915-924]. The most studied modification with a potential pathological significance is LDL oxidation [Aviram M. (1996) Europ. J. Clin. Chem. Clin. Biochem. 34:599-608; Aviram M. (1995) Isr. J. Med. Sci. 31:41-249; Chisolm G. M. and Steinberg D. (2000) Free Radic. Biol. Med. 28:1815-1826]. This modification leads to increased macrophage uptake of the modified lipoprotein, followed by cellular cholesterol accumulation that results with the formation of lipid-laden foam cells [Aviram (1996) id ibid.; Aviram (1995) id ibid.; Chisolm (2000) id ibid.; Aviram M. (1999) Antiox. Redox. Signal 1:585-594].

Several reports have implicated oxidative stress as the main factor triggering atherosclerosis [Heinecke, J. W. (2003) Am. J. Cardiol. 91:12A-16A; Ceconi, C. et al. (2003) Arch. Biochem. Biophys. 420:217-221; Dhalla, N. S. et al. (2000) J. Hypertens. 18:655-6731. Oxidative stress is defined as the result of an excess in free radicals (FR), which come in contact with cellular membranes and can lead to oxidative damage in biological molecules, such as lipids, carbohydrates, proteins and nucleic acids [Thomas C. E. and Aust, S. D. (1986) Ann. Emerg. Med. 15(9): 1075-83]. One of the molecules that may be attacked by FR is LDL, forming ox-LDL, whose high levels lead to atherosclerosis.

Increasing evidence in both experimental and clinical studies suggests that oxidative stress plays a major role in the pathogenesis of both types of diabetes mellitus. The possible sources for the overproduction of reactive oxygen species is widespread and include enzymatic pathways, autoxidation of glucose and the mitochondria. Abnormally high levels of these free radicals and the simultaneous decline of antioxidant defense mechanisms can lead to increased lipid peroxidation, damage of cellular organelles and enzymes and development of CVD. Thus, prevention of oxidative stress in diabetes is considered by many investigators to be a primary defense against the development of diabetic vascular disease. Moreover, some recent studies point at oxidative stress, activation of the sorbital pathway, advanced glycation endproducts (AGE), and AGE precursors, as the basic abnormalities that lead to the CVD in these patients, rather than hyperglycemia [Duckworth W. C. (2001) Curr. Atheroscler. Rep. 3:383-91; Yorek M. A. (2003) Free Radic. Res. 37:471-80; Maritim A. C. (2003) J. Biochem. Mol. Toxicol. 17:24-38.)

Paraoxonase (PON1) is an esterase, transported in the plasma as a component of HDL, associated to ApoAI and ApoJ. It has been shown in vitro that purified PON1 and HDL-associated PON1 inhibit the oxidative modification of LDL. Thus, the presence of PON1 in HDL may account for a proportion of the anti-oxidant properties of these lipoproteins [Tsuzura, S., et al. (2004) Metabolism 53:297-302]. Interestingly, several investigators have shown that serum paraoxonase activity is lower in diabetic patients and is lower yet in those with diabetic complications, independent of PON1 gene polymorphisms. These observations are consistent with in vivo increased oxidative stress levels in diabetic patients.

The LDL oxidation hypothesis of atherosclerosis raised an extensive investigation into the role of anti-oxidants against LDL oxidation as a possible preventive treatment of atherosclerosis. Although increased resistance of LDL to oxidation was observed after treatment with various synthetic pharmaceutical agents, an effort has been made to identify natural food products, which offer anti-oxidant defense against LDL oxidation.

Olive oil has been shown to inhibit LDL oxidation and this effect could be related to its high oleic acid content, as well as to some phenolics (hydroxytoluene, oleoropein) and phytosterols such as sitosterol [Aviram M. and Kasem E. (1993) Ann. Nutr. Metabol. 37:75-84; Visioli F. et al. (1995) Atherosclerosis 117:25-32].

In addition to LDL oxidation, a known risk factor for coronary heart disease (CHD)—the result of atherosclerosis in the coronary arteries—includes high serum LDL cholesterol concentration. There is a positive linear relationship between serum total cholesterol and LDL cholesterol concentrations, and risk of, or mortality from CHD [Jousilahtu et al. (1998) Circulation 97:1084-1094].

High concentrations of serum triacylglycerols may also contribute to CHD [Austin, M. A. (1989) Am. J. Epidemiol. 129:249-259], but the evidence is less clear. Diacylglycerols (DAG) have been shown to lower the postprandial elevation of serum triacylglycerols levels compared with triacylglycerols in healthy humans [Taguchi, H et al. (2000) J. Am. Coll. Nutr. 19:789-7961. Serum triglyceride (TG) concentrations after ingestion of 44 g of DAG oil were significantly low at six hours postprandially as compared to those after ingestion of 44 g of TG oil. The difference was reproducible even with low fat doses (10 and 20 g) [Moreau R. A., et al. (2002) Progress in Lipid Research 41:457-500].

Phytosterols and CHD

The term “phytosterols” covers plant sterols and plant stanols. Plant sterols are naturally occurring substances present in the diet as minor components of vegetable oils. Plant sterols have a role in plants similar to that of cholesterol in mammals, e.g. forming cell membrane structures. In human nutrition, both plant sterols and plant stanols are effective in lowering total plasma cholesterol levels and LDL-cholesterol.

The consumption of plant sterols and plant stanols lowers blood cholesterol levels by inhibiting the absorption of dietary and endogenously-produced cholesterol from the small intestine. The plant sterols/stanols are very poorly absorbable compounds. This inhibition is related to the similarity in physico-chemical properties of plant sterols and stanols to cholesterol.

The blood cholesterol-lowering effect of plant sterols has been investigated in a large number of clinical trials involving over 2,400 subjects, using doses as high as 25 grams per day for durations as long as three years. No significant adverse effects have been observed throughout the decades of medically supervised clinical efficacy testing or the general clinical use of plant sterols. Furthermore, the drug Cytellin (primarily β-sitosterol) was prescribed for more than 20 years and had an excellent safety record.

In addition, both plant sterols and plant stanols have been subjected to rigorous toxicological evaluation. Studies on the absorption, distribution, metabolism and excretion have shown that plant sterols are poorly absorbed from the intestine (1-10%).

A series of human studies with vegetable oil plant sterol esters in spreads, with intakes of up to 8.6 grams of plant sterols/ day for 4 weeks, has been conducted. Clinical chemistry, haematology, bacterial profiles of the gut microflora and general physical condition were evaluated. As in all other studies, no adverse effects were detected.

In the United States, a panel of independent experts has concluded that vegetable oil sterol esters, meeting appropriate food-grade specifications and produced by current good manufacturing practice (21 C.F.R. §182.1(b)), are safe for use as an ingredient in vegetable oil spreads, in amounts which do not exceed 20% of plant sterol esters. It was the Panel's opinion, together with qualified experts in the field, that vegetable oil sterol esters are safe for use, i.e. vegetable oil sterol esters were granted the GRAS status (Generally Recognized As Safe). Based on the GRAS recognition, the US Food and Drug Administration (FDA) has cleared to use a spread containing up to 20% of plant sterol esters and another one containing plant stanol ester. Similar approvals were given in different European countries as well as in Asia and Australia.

The role of diet in the promotion or prevention of heart disease is the subject of considerable research. However, the use of naturally-occurring materials which can lower LDL-cholesterol and triglycerides levels and inhibit LDL-oxidation should be advantageous over the use of synthetic drugs.

A recent review teaches that in recent years, with the growing interest in functional foods, the use of phytosterols for reducing serum cholesterol levels has gained considerable momentum [Stark, A. H. et al. (2002) Nutrition Reviews 60(6):170-176]. This should be attributed, inter alia, to the esterification of phytostanol with fatty acids (stanyl esters), providing commercial scale production of phytosterol-containing foods, such as margarines. Like stanyl esters, phytosteryl esters (steryl esters) have been shown in clinical studies to consistently lower serum LDL-cholesterol (LDL-C) levels (reducing by up to about 10% or more), with no change seen in HDL-cholesterol (HDL-C) values. The review suggests that properly formulated free phytosterols and stanols may be as effective as stanyl and steryl esters in lowering LDL-C levels in humans.

WO 01/32035 teaches olive oil-based products, based on especially higher grades of olive oils (such as virgin olive oils), comprising plant stanol esters and/or plant sterol esters.

U.S. Pat. No. 5,843,499 discloses oil extractable from corn fiber that contains ferulate esters (phytosterol esters which are esterified to ferulic acid), in particular sitostanyl ester, which has been shown to have cholesterol-lowering activity. It is mentioned that corn fiber oil (containing about 73% fat (triacylglycerol), 8% sterol (fatty acyl) esters, 4% free sterols, 6% diacylglycerols and 6% ferulate (sterol esters)) is used as an additive to supplementary food for reducing cholesterol level.

U.S. Pat. No. 6,326,050 discloses a composition consisting of oil or fat, a diacylglycerol, a free phytosterol and tocopherol, dissolved or dispersed in the oil or fat. This composition plays a role in lowering blood cholesterol of hypercholesterolemic individuals. None of the above mentioned publications describes reduction of both cholesterol and triglycerides serum levels.

Olive oil, in contrast to other mentioned vegetable oils (such as corn fiber oil, table cooking oil, soybean oil, rapeseed oil, rice bran oil, and palm oil) is composed, inter alia, of 55 to 85% monounsaturated fatty acids (MUFA), in particular oleic acid, which contribute to the high nutritional value of this oil. There are some distinct advantages of using olive oil over other vegetable oils. Diets rich in olive oil have been shown to be more effective in lowering total cholesterol and LDL-cholesterol than conventional dietary treatments not containing high levels of MUFA [Brown M. S and Goldstein J. L. (1983) Ann. Rev. Biochem. 52:223-261].

Furthermore, olive oil is an integral ingredient of the Mediterranean diet and accumulating data suggests that it may have health benefits that include reduction of risk factors of coronary artery disease, prevention of several types of cancer, and modification of immune and inflammatory response [Brown and Goldstein (1983) id ibid.].

Co-owned WO03/064444 describes a composition of matter comprising diacylglycerol(s), mainly 1,3-diacylglycerols (DAG) and phytosterol and/or phytostanol ester(s) (PSE), dispersed in oil and/or fat.

In search for a combined-effect nutritional supplement and/or pharmaceutical, the inventors have now found that the said combination of diacylglycerol(s), mainly 1,3-DAGs, and PSEs, preferably dissolved or dispersed in oil and/or fat, has a synergistic effect and decreases both LDL-cholesterol and triglycerides levels in the blood. This combination may further exhibit increased serum and macrophage anti-oxidative properties, and in particular LDL anti-oxidative properties, resulting in reduction of the risk for CHD and arteriovascular diseases. These novel therapeutic uses are major objects of the present invention.

It is a further object of the present invention to reduce the levels of blood cholesterol and triglycerides, and consequently the risk for CHD in mammals, by administration of the said combination, which was found to decrease blood cholesterol levels to a greater extent compared to the combined reduction of cholesterol and triglycerides levels obtained by using each of said two ingredients (DAG and PSE) separately in oil. Such synergistic effect has not been described or demonstrated in the prior art. As mentioned, U.S. Pat. No. 6,326,050 refers to a combination of diacylglycerols and free phytosterols. Although it is stated in this patent that when the amount of diacylglycerols is over 80wt % a synergistic effect on lipid metabolism may be expected, no such effect is demonstrated or discussed in the patent.

In this respect it may be emphasized that according to the present invention, an effect in reducing both cholesterol and triglycerides serum levels, together with increased anti-oxidative properties, is observed even when a combination containing only 11 wt % DAG and 20 wt % phytosterol esters (in oil) is employed.

It is a further object of the present invention to use said combination as a dietary/nutritional supplement (food additive) or in pharmaceutical preparation form.

These and other objects of the invention will become apparent as the description proceeds.

### SUMMARY OF THE INVENTION

The present invention relates to a method for reducing blood levels of both cholesterol and triglycerides and/or for lowering serum, serum LDL and macrophage oxidation levels, inhibiting the formation of foam cells and/or preventing the deleterious effects generated by lipid-induced oxidative stress in a subject in need, comprising administering to said subject a dietary nutrient or food supplement comprising an edible combination of diacylglycerol(s) (DAG), mainly 1,3-diacylglycerol(s), and phytosterol and/or phytostanol ester(s) (PSE), wherein the weight ratio of PSE:DAG in said combination is at least 1.

In this and all other methods of the invention, in a preferred embodiment the said combination of DAG and PSE is dissolved or dispersed in an edible oil and/or fat.

The said oil may be a natural and/or edible oil, preferably olive oil, soybean oil, sunflower oil, safflower oil, canola oil, palm oil, avocado oil, sesame oil and fish oil, more preferably olive oil, canola oil or fish oil.

The said fat may be any natural fat, preferably butter fat, anhydrous milk fat, cocoa butter or lard.

The fatty acid residues of the DAG and the PSE preferably correspond to the fatty acid residues of the oil from which it is derived, such as oleic, palmitic, palmitoleic, stearic, linoleic, linolenic, and eicosanoic acid residues.

The phytosterol ester(s) is/are fatty acid ester(s) of, for example, stigmasterol, sitosterol, beta-sitosterol, brassicasterol, campesterol and/or 5-avenasterol and isomers and derivatives thereof and said phytostanol ester(s) is/are fatty acid ester(s) of, for example, beta-sitostanol, campestanol and/or stigmastanol and isomers and derivatives thereof.

The combination used by the method of the invention may further comprise physiologically compatible, particularly edible, conventional ingredients of nutritional compositions.

In a specific embodiment, weight ratio between phytosterol and/or phytostanol esters and diacylglycerol(s) and phytosterol and/or phytostanol ester(s) in said combination may be from about 15:1 to 1:1, preferably about 10:1 to about 1:1, more preferably 5:1 to 1:1 and particularly about 2:1.

The amount of diacylglycerol(s) in said combination is preferably at least 1 wt %.

The amount of phytosterol and/or phytostanol ester(s) in said combination is preferably at least 1 wt %.

In further particular embodiments, the amount of diacylglycerol(s) in said combination is from about 1 to about 99 wt %, preferably from about 4 to about 70 wt %, particularly from about 7 to about 48 wt %, and the amount of phytosterol and/or phytostanol ester(s) in said combination is from about 1 to about 99 wt %, preferably from about 5 to about 70 wt %, more particularly from about 10 to about 60 wt %, specifically from about 7 to about 35 wt % and more specifically from about 20 to about 35 wt %.

In another particular embodiment, the said combination consists of 15 wt % DAG, mainly 1,3-DAG(s) and 25 wt % total PSE(s) dissolved or dispersed in any one of olive, canola and fish oil.

In a further aspect, the invention relates to a method for treating and/or preventing cardiovascular disorders and diseases related thereto, particularly coronary heart disease, atherosclerosis and cardiovascular disorders induced or manifested by other diseases such as metabolic syndrome and any of the conditions involved therein, particularly diabetes mellitus and more particularly Type II diabetes, in a subject in need, comprising administering to said subject a dietary nutrient or food supplement comprising an edible combination of diacylglycerol(s) (DAG), mainly 1,3-diacylglycerol(s), and phytosterol and/or phytostanol ester(s) (PSE), wherein the weight ratio of PSE:DAG in said combination is at least 1. Also in this method, the said combination of DAG and PSE is preferably dissolved or dispersed in an edible oil and/or fat.

Still further, the invention relates to a method for reducing blood levels of both cholesterol and triglycerides and/or for lowering serum, serum LDL and macrophage oxidation levels, inhibiting the formation of foam cells and/or preventing the deleterious effects generated by lipid-induced oxidative stress in a subject in need, comprising administering to a subject an orally administrable pharmaceutical composition comprising a combination of diacylglycerol(s) (DAG), mainly 1,3-diacylglycerol(s) and phytosterol and/or phytostanol ester(s) (PSE), wherein the weight ratio of PSE:DAG in said combination is at least 1, and further comprising pharmaceutically acceptable additives, diluents, excipients and/or carriers.

This method of the invention may be particularly intended for the treatment and/or prevention of cardiovascular disorders and diseases related to the same, coronary heart disease and/or atherosclerosis and cardiovascular disorders induced or manifested by other diseases particularly diabetes mellitus, and more particularly Type II diabetes.

In a particular embodiment, the invention relates to a method for reducing blood levels of cholesterol and triglycerides and/or reducing LDL oxidation in a subject in need, comprising administering to said subject a pharmaceutical composition consisting substantially of 15 wt % DAG(s), mainly 1,3-DAG(s) and 25 wt % total PSE(s) dissolved or dispersed in olive oil, wherein said composition is characterized by having the effect of reducing blood levels of both cholesterol and triglycerides and/or for lowering serum, serum LDL and macrophage oxidation levels, inhibiting the formation of foam cells and/or preventing the deleterious effects generated by lipid-induced oxidative stress.

In addition, the invention relates to a method for maintaining PON1 activity levels in a patient in need, thereby reducing and/or preventing LDL oxidation and attenuating CVD and/or susceptibility of diabetic patients to vascular complications, comprising administering to said patient a dietary nutrient or food supplement or a pharmaceutical composition comprising an edible combination of diacylglycerol(s) (DAG), mainly 1,3-diacylglycerol(s), and phytosterol and/or phytostanol ester(s) (PSE), wherein the ratio of PSE:DAG in said combination is at least 1.

The invention also relates to a method for maintaining Lp(a) levels and reducing and/or preventing the extent of angiographically documented Coronary Artery Disease in a subject in need comprising administering to said patient a dietary nutrient or food supplement or a pharmaceutical composition comprising an edible combination of diacylglycerol(s) (DAG), mainly 1,3-diacylglycerol(s), and phytosterol and/or phytostanol ester(s) (PSE), wherein the ratio of PSE:DAG in said combination is at least 1.

In yet a further aspect, the invention relates to a dietary nutrient or food supplement comprising an edible composition for reducing blood levels of both cholesterol and triglycerides and/or for lowering serum, serum LDL and macrophage oxidation levels, inhibiting the formation of foam cells and/or preventing the deleterious effects generated by lipid-induced oxidative stress, said composition comprising a combination of diacylglycerol(s) (DAG), mainly 1,3-diacylglycerol(s) and phytosterol and/or phytostanol ester(s) (PSE) wherein the ratio of PSE to DAG in said combination is at least 1, optionally further comprising conventional ingredients of nutritional compositions.

The combination comprised in the dietary nutrient of the invention is preferably dissolved or dispersed in an edible oil and/or fat.

In the dietary nutrient or food supplement of the invention said oil may be a natural and/or edible oil, preferably olive oil, soybean oil, sunflower oil, safflower oil, canola oil, palm oil, avocado oil, sesame oil or fish oil, more preferably olive oil, canola oil or fish oil. The said fat may be any natural fat, preferably butter fat, anhydrous milk fat, cocoa butter or lard.

In the dietary nutrient or food supplement of the invention, the fatty acid residues of the DAG may correspond to the fatty acid residues of the oil from which it is derived, such as oleic, palmitic, palmitoleic, stearic, linoleic, linolenic, and eicosanoic acid residues.

In the dietary nutrient or food supplement of the invention the phytosterol ester(s) is/are fatty acid ester(s) of for example stigmasterol, sitosterol, beta-sitosterol, brassicasterol, campesterol and/or 5-avenasterol and isomers and derivatives thereof and said phytostanol ester(s) is/are fatty acid ester(s) of for example beta-sitostanol, campestanol and/or stigmastanol and isomers and derivatives thereof.

In the dietary nutrient or food supplement of the invention the weight ratio between phytosterol and/or phytostanol ester(s) and between diacylglycerol(s) in said combination may be from about 15:1 to 1:1, preferably about 10:1 to about 1:1, more preferably 5:1 to 1:1 and particularly about 2:1.

The amount of diacylglycerol(s) in the combination/composition comprised in dietary nutrient or food supplement of the invention is preferably at least 1 wt % and the amount of phytosterol and/or phytostanol ester(s) is at least 1 wt %.

In the dietary nutrient or food supplement of the invention the amount of diacylglycerol(s) in said combination is from about 1 to about 99 wt %, preferably from about 4 to about 70 wt/o, particularly from about 7 to about 48 wt %, more particularly from about 10 to about 22 wt % and the amount of phytosterol and/or phytostanol ester(s) in said combination is from about 1 to about 99 wt %, preferably from about 5 to about 70 wt %, more particularly from about 7 to about 60 wt %, specifically from about 10 to about 60 wt %, more particularly from about 7 to about 35 wt % and more specifically from about 20 to about 35 wt %.

In a particular embodiment, the said combination/composition comprised in the dietary nutrient or food supplement of the invention consists of 15 wt % DAG, mainly 1,3-DAG(s) and 25 wt % total PSE(s) dissolved or dispersed in any one of olive, canola and fish oil.

The invention will be described in more detail on hand of the accompanying Figures.

### BRIEF DESCRIPTION OF THE FIGURES

FIG. 1A-B: The effect of olive oil, olive oil+phytosterols, and PSE+DAG in olive oil on macrophage cellular peroxides content

FIG. 1A: Macrophage peroxide levels determined by the cells mean fluorescence (emitted by DCF) intensity.

FIG. 1B: Macrophage peroxide levels determined by the percentage of fluorescent positive cells.

Abbreviations: PS-E, Plant sterols esters; DAG, diacylglycerol, cont., control; Ol. O., olive oil.

FIG. 2: The effect of olive oil, olive oil+phytosterols, and PSE+DAG in olive oil on macrophage superoxides anions release

Macrophage superoxide ions release was determined by the superoxide dismutase-inhibitable reduction of cytochrome C.

Abbreviations: PS-E, Plant sterols esters; DAG, diacylglycerol, cont., control; Ol. O., olive oil.

FIG. 3: Effect of PSE+DAG in canola oil consumption on serum triglycerides profile

FIG. 4: Effect of PSE+DAG in canola oil consumption on serum total cholesterol profile

FIG. 5: Effect of PSE+DAG in canola oil and PSE+DAG in fish oil consumption on serum oxidative stress

FIG. 6: Effect of PSE+DAG in canola oil and PSE+DAG in fish oil consumption on serum PON1 activity

Abbreviations: Ser. PON1 act., serum PON1 activity; Plac., placebo; Can., canola.

FIG. 7: Effect of PSE+DAG in canola oil and PSE+DAG in fish oil consumption on ox-LDL uptake by peritoneal macrophages

Abbreviations: deg., degradation; ass., association.

FIG. 8: Effect of PSE+DAG in canola oil and PSE+DAG in fish oil consumption on macrophage oxidative status

FIG. 9: Effect of PSE+DAG in canola oil and PSE+DAG in fish oil consumption on PMA-induced superoxide anions release in macrophages

FIG. 10: Change in total cholesterol concentrations

Hypercholesterolemic overweight volunteers were fed for four weeks with control olive oil diet or PS-E+DAG in olive oil followed by four weeks of washout and counter supplementation. Total cholesterol levels were tested at the beginning (open squares) and at the termination (closed squares) of each phase as described in methods. Values represent mean±SEM of the total cholesterol concentrations in 21 patients. Statistical significance between baseline and endpoint values as found by Student's t-test is *P<0.05 or **P<0.001.

FIG. 11: Change in LDL cholesterol concentrations

Hypercholesterolemic overweight volunteers were fed for four weeks with control olive oil diet or PS-E+DAG in olive oil followed by four weeks of washout and counter supplementation. LDL cholesterol levels were tested at the beginning (open squares) and at the termination (closed squares) of each phase as described in methods. Values represent mean±SEM of the LDL cholesterol concentrations in 21 patients. Statistical significance between baseline and endpoint values as found by Student's t-test is *P<0.10 or **P<0.001.

FIG. 12: Change in Apolipoprotein B concentrations

Hypercholesterolemic overweight volunteers were fed for four weeks with control olive oil diet or PS-E+DAG in olive oil followed by four weeks of washout and counter supplementation. ApoB levels were tested at the beginning (open squares) and at the termination (closed squares) of each phase as described in methods. Values represent mean±SEM of the apoB concentrations in 21 patients. Statistical significance between baseline and endpoint values as found by Student's t-test is *P<0.05 or **P<0.001.

FIG. 13: Change in ApoB to ApoA ratio

Hypercholesterolemic overweight volunteers were fed for four weeks with control olive oil diet or PS-E+DAG in olive oil followed by four weeks of washout and counter supplementation. ApoB/ApoA ratios were tested at the beginning (open squares) and at the termination (closed squares) of each phase as described in methods. Values represent mean±SEM of the ApoB/ApoA ratios in 21 patients. Statistical significance between baseline and endpoint values as found by Student's t-test is *P<0.05.

FIG. 14: Change in lipoprotein(a) concentrations

Hypercholesterolemic overweight volunteers were fed for four weeks with control olive oil diet or PS-E+DAG in olive oil followed by four weeks of washout and counter supplementation. Lp(a) levels were tested at the beginning (open squares) and at the termination (closed squares) of each phase as described in methods. Values represent mean±SEM of the Lp(a) concentrations in 21 patients. Statistical significance between baseline and endpoint values as found by Student's t-test is * P<0.01.

FIG. 15: Change in serum lipid peroxides concentrations

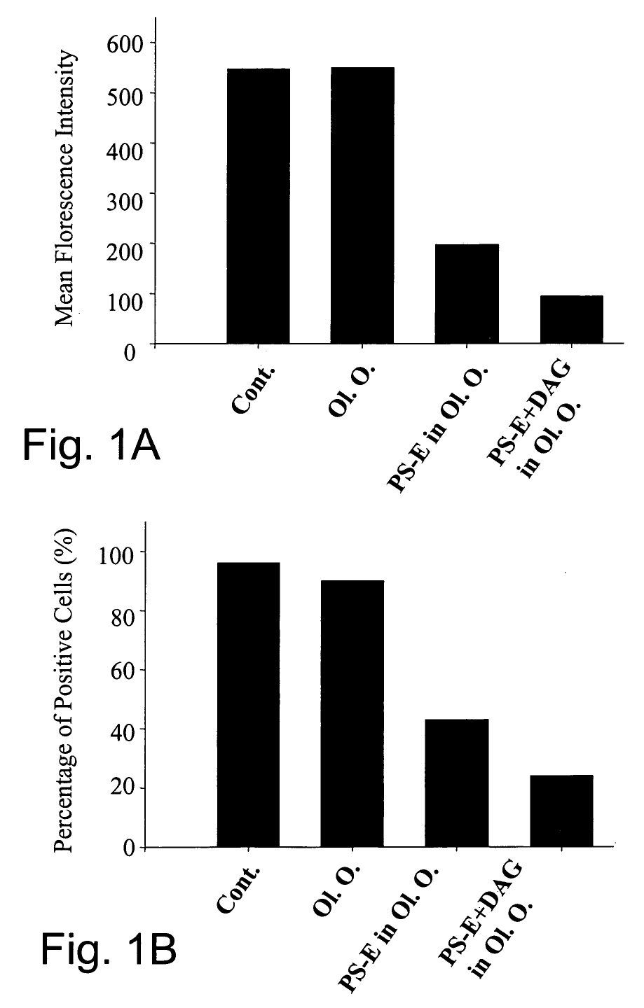

Hypercholesterolemic overweight volunteers were fed for four weeks with control olive oil diet or PS-E+DAG in olive oil followed by four weeks of washout and counter supplementation. TBARS levels were tested at the beginning (open squares) and at the termination (closed squares) of each phase as described in methods. Values represent mean±SEM of the TBARS concentrations in 21 patients. Statistical significance between baseline and endpoint values as found by Student's t-test is *P<0.10 or **P<0.01.

### DETAILED DESCRIPTION OF THE INVENTION

The following abbreviations and definitions are used along the specification:
Canola composition: PSE+DAG in a canola oil base.
CHD: Coronary heart disease
CVD: Cardiovascular disease
DAG: Diacylglycerol(s), mainly 1,3-diacylglycerols
DCF Dichlorofluorescin
Fish oil composition: PSE+DAG in a fish oil base.
HBSS: Hanks' Balanced Salts Solution
HDL: High density lipoproteins
LDL: Low density lipoproteins
MPM: Mouse peritoneal macrophages
MUFA: Monounsaturated fatty acid
Olive oil composition: PSE+DAG combination in an olive oil base.
Ox-LDL: Oxidized LDL
PMA: Phorbol myristate acetate
PSE+DAG combination: A combination of diacylglycerol(s) (DAG), mainly 1,3-diacylglycerol(s) and phytosterol and/or phytosterol esters (PSE)
PSE+DAG composition: A combination of diacylglycerol(s) (DAG), mainly 1,3-diacylglycerol(s) and phytosterol and/or phytosterol esters (PSE) in an oil and/or fat base.
PBS: Phosphate Buffered Saline
PSE: Phytosterol or phytostanol ester(s)
RDA: Recommended daily allowance.

The present inventors used the animal model system apoE⁰ mice, in which severe hypercholesterolemia and atherosclerotic plaques are generated at an early age to evaluate the anti-atherosclerotic properties of novel edible compositions, herein referred to as PSE+DAG-(olive), PSE+DAG-(canola) and PSE+DAG-(fish), in comparison with placebo and/or with canola oil.

As mentioned above, the inventors found that a combination of DAGs, mainly 1,3-DAGs and PSE in oil and/or fat, provides an enhanced effect, by decreasing both LDL-cholesterol and triglycerides levels in the blood. This combination, and compositions comprising the same, further exhibits increased serum, serum LDL and macrophage anti-oxidative properties, as well as inhibiting the formation of foam cells and/or preventing the deleterious effects generated by lipid-induced oxidative stress, which result in reduction of the risk for CHD and arteriovascular-related diseases, like, e.g., diabetes. These results were confirmed in a human study, as presented below.

The invention thus mainly relates to a novel use of a combination comprising diacylglycerols and phytosterol and/or phytostanol ester(s) as an agent capable of reducing blood levels of both cholesterol and triglycerides and/or for lowering serum, serum LDL and macrophage oxidation levels, inhibiting the formation of foam cells and/or preventing the deleterious effects generated by lipid-induced oxidative stress.

As demonstrated in the Examples and in the Figures, the various PSE+DAG combinations were capable of substantially reducing the levels of blood triglycerides and cholesterol, compared to a placebo treatment, in the animal model system.

The combination used by the present invention consists essentially of phytosterol and/or phytostanol ester(s) and diacylglycerol(s), mainly 1,3-diacylglycerols, preferably dispersed in an edible oil and/or fat, wherein the ratio of PSE to DAG is at least 1. More particularly, the combination comprises at least 1 wt % diacylglycerol(s) and at least 1 wt % phytosterol and/or phytostanol ester(s) dissolved or dispersed in said oil and/or fat.

The amount of diacylglycerol(s) contained in the oil and or fat may range from 1 wt % to about 99 wt %, preferably from about 7 wt % to about 48 wt % and most preferably from about lOwt % to about 22 wt %.

The amount of phytosterol and/or phytostanol ester(s) contained in the oil may range from from about 1 to about 99 wt %, preferably from about 5 to about 70 wt %, more particularly from about 7 to about 60 wt %, specifically from about 10 to about 60 wt %, more particularly from about 7 to about 35 wt % and more specifically from about 20 to about 35 wt %.

The diacylglycerol(s) consist substantially of 1,3-diacylglycerol(s). The fatty acid profile of the diacylglycerol(s) depends on the particular oil and/or fat used for producing the PSE and DAG combination. For example, when olive oil is used, the diacylglycerols mainly consists of 1,3-dioleyl glycerol. Generally speaking, fatty acid moieties of the DAG may include, for example, caproic, caprylic, myristic, oleic, palmitic, palmitoleic, stearic, linoleic, linolenic, and eicosanoic fatty acids, but may contain other fatty acyls.

The phytosterol and/or phytostanol moiety of the corresponding esters of the combination of the invention may be any phytosterol or phytostanol derivative. These phytosterols and/or phytostanols can be from soy, wood, algae, and other plants, as well as from animal or microbial sources that contain phytosterols and/or phytostanols. Examples of phytosterols and/or phytostanols include but are not limited to beta-sitosterol, stigmasterol, campesterol, brassicasterol, beta-sitostanol, campestanol, and stigmastanol.

The fatty acid moiety of the fatty acid phytosterol and/or phytostanol esters of the invention may include any fatty acid and more preferably different fatty acids characterizing the source of the fatty acids. Such source may be a natural, synthetic, or fractionated oil or fat used in the interesterification of said phytosterols and/or phytostanols. In other examples the source of the fatty acids may be any other esters of fatty acids, such as alkyl esters of fatty acids, preferably ethyl or methyl esters, or free fatty acids. The fatty acids may include, but are not limited to, caproic, caprylic, myristic, oleic, palmitic, palmitoleic, stearic, linoleic, linolenic, and eicosanoic fatty acids. It should be emphasized that in a preferred embodiment the combination of the invention is produced by the in situ esterification, chemical or enzymatic, of a preferred oil and/or fat with a source of phytosterols and/or phytostanols, simultaneously yielding diglycerides (DAG) and thus obtaining the combination of the invention in which the fatty acid profile of the DAG and the fatty acids of the sterol or stanol esters corresponds to the fatty acid profile of the preferred oil and/or fat used for the interesterification. In this process, the resulting combination is dissolved or dispersed in the source oil and/or fat and can optionally be further purified. The combination of the invention may also be obtained by separately producing phytosterol and/or phytostanol esters with a preferred source of fatty acids and combining said phytosterol and/or phytostanol esters with DAG. The latter can be commercially available or produced by conventional chemical or enzymatic processes, including hydrolysis, alcoholysis, transesterifications or interesterification processes. The fatty acid profile of the DAG of the combination of the invention corresponds to the fatty acid profile of the source oil and/or fat used in their preparation.

The weight ratio of phytosterol and/or phytostanol ester(s) to diacylglycerol(s) in the combination or composition of the invention is at least 1:1. This weight ratio may be from about 15:1 to about 1:1, preferably about 10:1 to about 1:1, more preferably 5:1 to 1:1 and particularly about 2:1. As used in the context of this application, ratios between various constituents are to be taken as weight ratios, unless specified otherwise.

The oil comprised in the composition of the invention may be any edible oil, including, but not limited to olive oil, soybean oil, sunflower oil, safflower oil, canola oil, sesame oil, palm oil, avocado oil or fish oil. Preferably the oil is at least one of olive oil, canola oil or fish oil. The fat contained in the composition of the invention may be any suitable fat, such as, e.g., butter fat, anhydrous milk fat, cocoa butter, as well as animal fat such as lard or a fish oil concentrate.

The diacylglycerol(s) may be obtained by any conventional enzymatic or non-enzymatic procedure. Preferably, they are obtained by inter-esterification reaction between phytosterol(s) and triglyceride(s) present in the oil and/or fat. The phytosterol and/or phytostanol ester(s) may be obtained by any conventional enzymatic or non-enzymatic procedure. Preferably, these constituents are obtained by interesterification reaction between phytosterol(s) and/or phytostanol and triglyceride(s) present in an edible oil or fat. A process for obtaining the combinations used by the present invention is described in detail in WO03/064444, fully incorporated herein by reference. As described in WO03/064444, the composition of the present invention may be also prepared by mixing (or blending) the desired amounts of diacylglycerol(s) and phytosterol and/or phytostanol ester(s) with the oil and/or fat.

As shown in the following examples, a significant effect of the tested composition in preventing and/or reducing serum ox-LDL, as well as macrophage oxidation was obtained. Thus, in addition to having an effect on reduction of blood LDL-cholesterol and triglycerides levels, the said combination, and compositions comprising the same, exhibit serum LDL and macrophage anti-oxidative properties. The examples further show that preparation of PSE+DAG in olive oil, canola oil and/or fish oil exhibited significant anti-oxidative properties, inhibiting the formation of foam cells, and/or preventing the deleterious effects generated by lipid-induced oxidative stress.

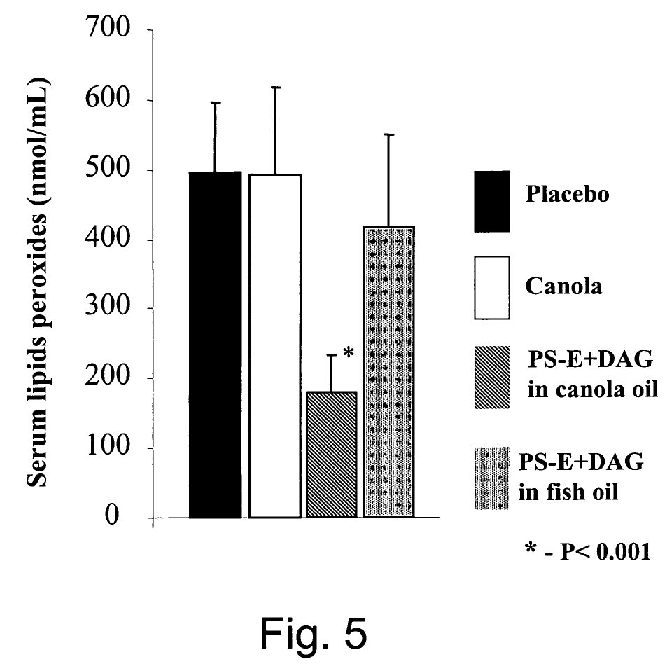

Specifically, FIGS. 5, 7, 8 and 9 demonstrate how the different PSE+DAG preparations lowered the following oxidative stress parameters: serum oxidative stress, ox-LDL uptake by peritoneal macrophages, macrophage oxidative status, and PMA-induced superoxide anions release from macrophages.

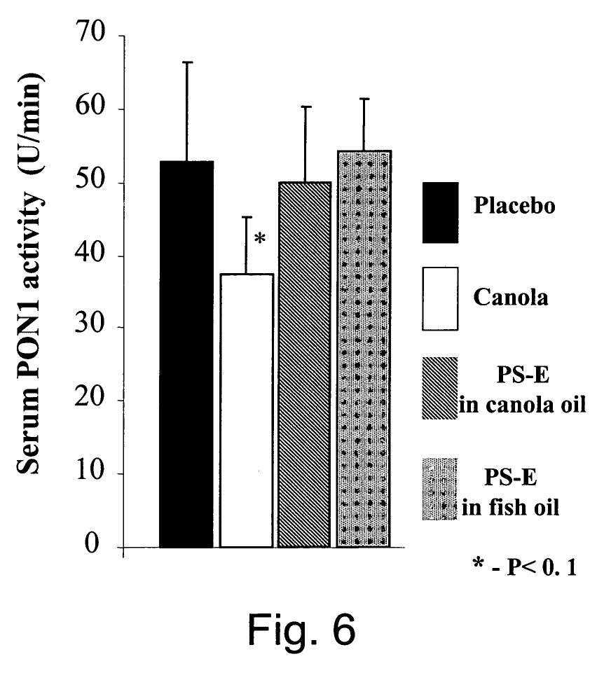

PON1 activity in the serum was also measured. PON1 is an HDL-associated esterase that can eliminate ox-LDL. Interestingly, the results of FIG. 6 show that while canola oil reduces PON1 activity, the PSE+DAG combinations of the invention were able to maintain PON1 higher levels, suggesting that the combination of the present invention may protect PON1 activity in a pro-atherosclerotic environment.

The efficacy of the combinations and compositions of the invention was then tested in humans. Well known, diet is the cornerstone of the prevention and treatment of CVD. Currently, National Cholesterol Education Program/American Heart Association Step I or Step II diets are typically recommended for lowering blood cholesterol concentrations. The primary objective of these diets is to lower saturated fat (8-10% and <7% of energy, respectively), cholesterol (300 or 200 mg/d), and total fat (<30% of energy) [Expert Panel on Detection, Evaluation, and Treatment of High Blood Cholesterol in Adults (2001) J.A.M.A. 285:2486-2497]. Typically, a Step I diet lowers total cholesterol and LDL cholesterol by <5-7%. A Step II diet can lower total cholesterol and LDL cholesterol an additional 3-7% [Stone N. J., et al. (1996) Circulation 94:3388-3391]. In these diets, saturated fat energy is replaced by carbohydrate, resulting in a low-fat, high-carbohydrate diet. Although these diets have beneficial effects on total cholesterol and LDL cholesterol, they increase plasma triglycerides concentrations and decrease HDL-cholesterol concentrations, thereby potentially adversely affecting CVD risk. This has caused some to question whether a Step I or Step II diet is the ideal diet for maximally reducing CVD risk [Katan M. B., et al. (1997) N. Engl. J. Med. 337:563-566; Connor W. E. and Connor S. L. (1997) N. Engl. J. Med. 337:566-567].

The alternative diet that has attracted much attention recently is a high-monounsaturated fatty acid (MUFA), cholesterol-lowering diet, in which saturated fat energy is replaced by MUFAs, resulting in a diet higher in total fat (i.e., >30% of energy) than a Step I or Step II diet. In contrast with a Step I or Step II diet, a high-MUFA diet does not raise triglycerides or lower HDL-cholesterol concentrations. To date, the primary food source of MUFAs that has been used is olive oil; canola oil has also been used. Little is known about how other food sources of MUFAs, such as peanuts, might affect the plasma lipid response to a cholesterol-lowering diet. Establishing the efficacy of other MUFA sources is important because it will increase the food options available in planning high-MUFA, cholesterol-lowering diets. Accordingly, flexibility in diet planning may enhance compliance with a cholesterol-lowering diet. Kris-Etherton and colleagues compared step II diet with MUFA enriched diets, in which olive oil was one of the sources and found it to lower total and LDL-cholesterol and triglycerides, but demonstrated no effect on HDL-cholesterol [Kris-Etherton P. M., et al., (1999) Am. J. Clin. Nutr. 70:1009-1015].

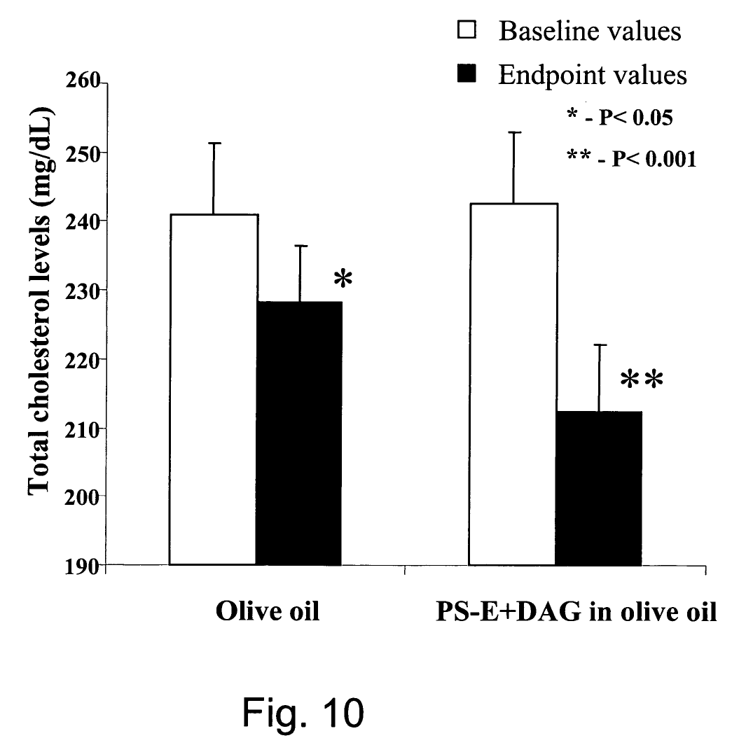

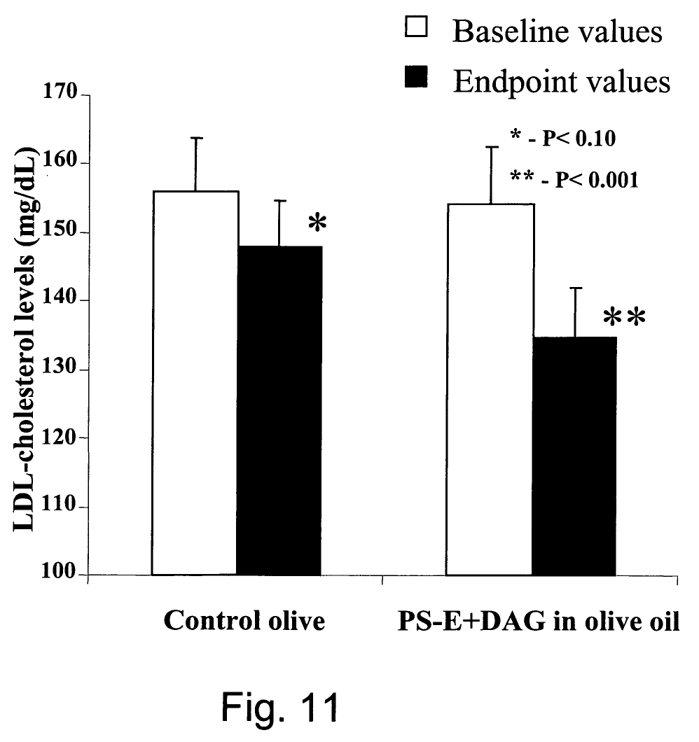

Indeed, as proved by the results of Example 3, the combinations/compositions of the invention are most effective in reducing total cholesterol (see FIG. 10) and LDL cholesterol plasma levels (see FIG. 11) as well as fasting triglycerides levels (see text below).

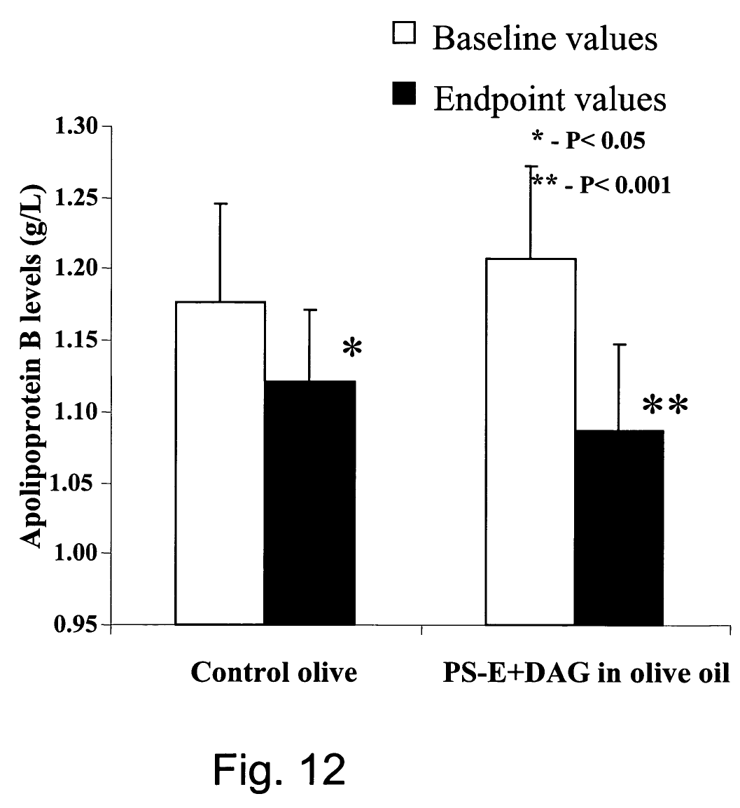

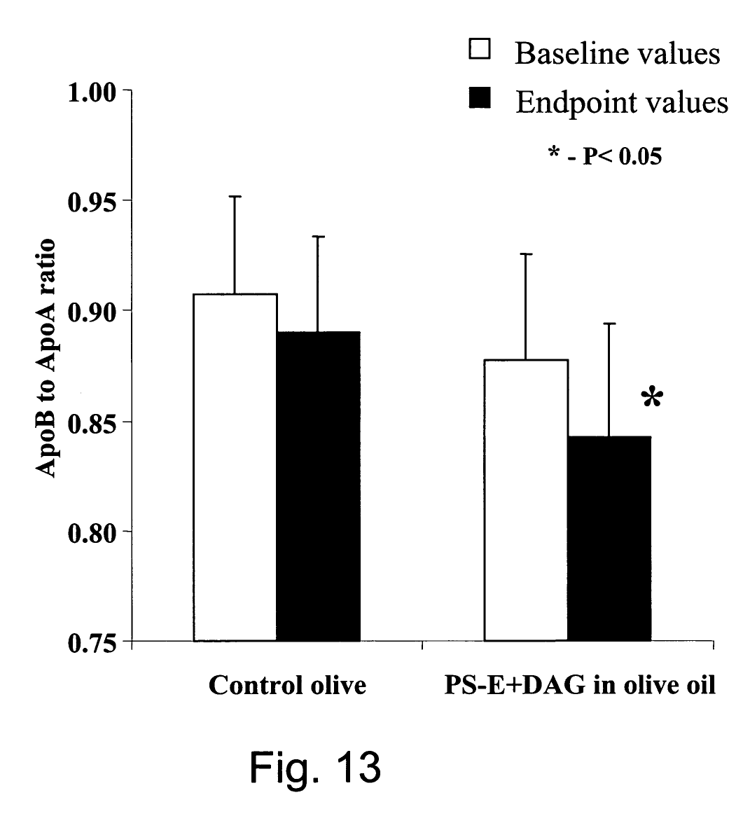

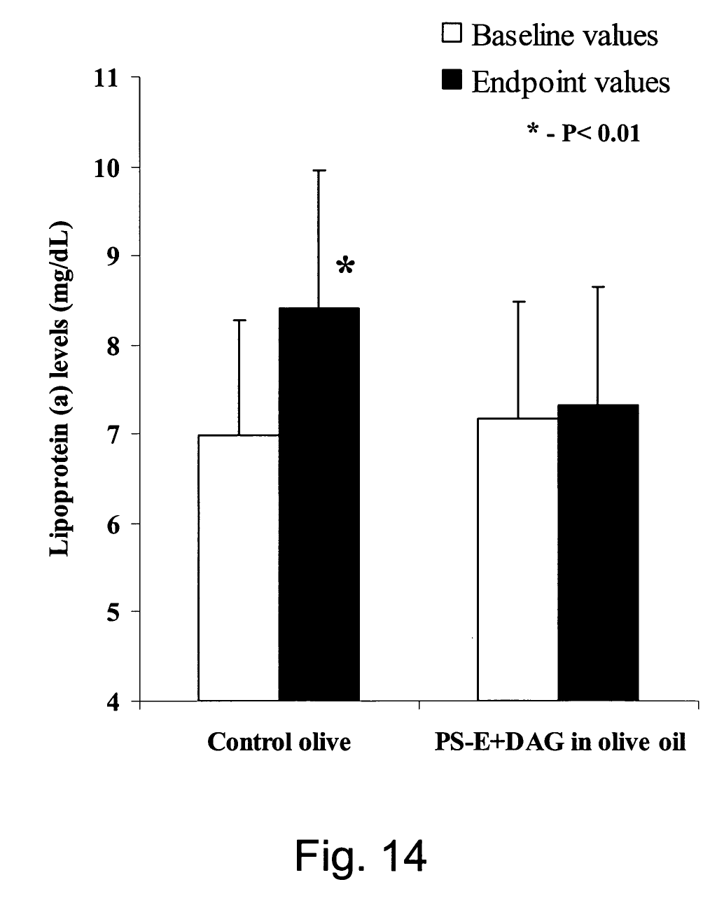

In addition, subjects who consumed the combination/composition of the invention showed a pronounced reduction of apo B values (FIG. 12). Consequently, there was also a positive change, i.e. decrease, in apoB/apoA ratio. Apo B, apo A-I and the apo B/apo A-I ratio have been reported as efficient predictors of cardiovascular events than LDL-C and as may be seen from FIG. 13, in addition to the aforementioned reduction in apo B levels, introducing PSE+DAG to the base MUFA-enriched diet consumed by the volunteers resulted in significantly reduced levels of apoB/apoA ratios. Lipoprotein(a) (Lp(a)) is an LDL-like particle to which apo A is attached through a disulfide bond to apo B. Increased plasma levels of Lp(a) are independent predictor of the presence of angiographically documented and clinical CAD, particularly in patients with hypercholesterolemia [Danesh J., et al. (2000) Circulation 102:1082-1085]. In the present human study, hypercholesterolemic volunteers fed with control diet presented significantly elevated levels of Lp(a) (FIG. 14).

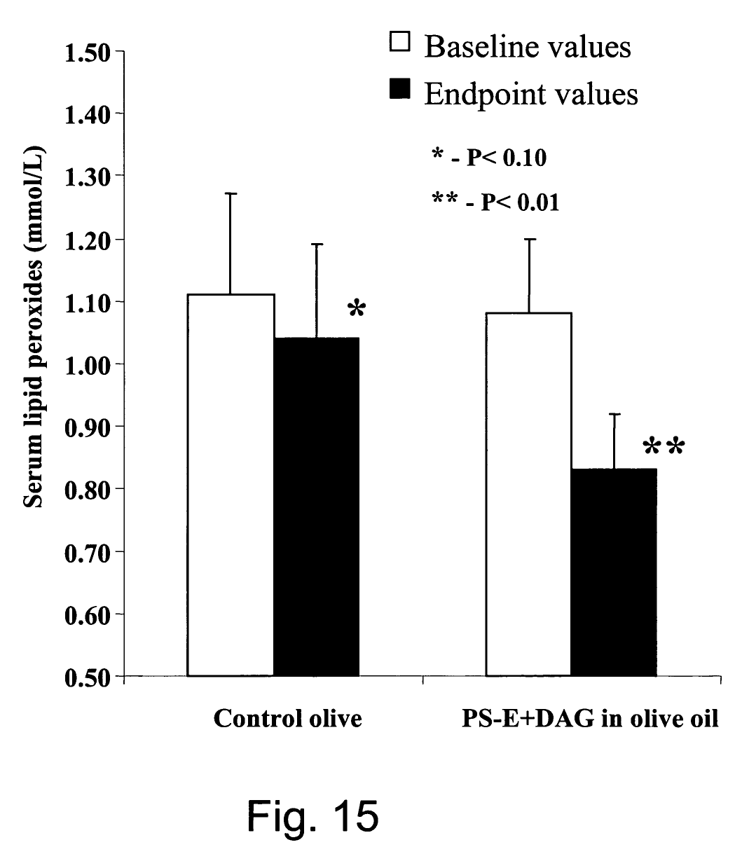

The present human study also demonstrates a pronounced and significant reduction in plasma oxidative stress levels (FIG. 15).

The PSE+DAG combinations may be used per se, as a food article. Such food article may be any conventional food, and also a functional food or beverage. Alternatively, the combinations of the invention may be an ingredient of a food article or a food supplement, which may further optionally contain conventional additives used in the food industry, such as preserving agents, colorants, flavoring agents, fragrances, antioxidative and hardening agents, vitamins, calcium, other minerals trace elements, probiotic agents, isoflavons, caloric agents and the like.

The terms dietary nutrient and food supplement may be used herein alternatively, and are to be taken to mean any edible supplements, particularly dietary supplements to edible products, preferably food articles, including functional foods and functional beverages. In particular, the combination of the invention may be used per se, for example in capsules, as a dietary supplement. Alternatively, the combination or composition of the invention may be used as an active ingredient/food additive of functional foods and beverages. Thus, the combination and compositions of the invention may be an ingredient of nutraceuticals. The food supplement of the invention may thus be introduced into the food, food article, drink or nutraceutical by admixing, adding or incorporating it during manufacture thereof.

Alternatively, the combination may be comprised as the active or auxiliary ingredient of a pharmaceutical composition for reducing blood levels of both cholesterol and triglycerides and/or for lowering oxidation levels of serum, serum LDL and macrophage oxidation, inhibiting the formation of foam cells and/or preventing the deleterious effects generated by lipid-induced oxidative stress. Pharmaceutical compositions may contain pharmaceutically acceptable additives, diluents, excipients and carriers.

The preparation of pharmaceutical compositions is well known in the art, see e.g., U.S. Pat. Nos. 5,736,519, 5,733,877, 5,554,378, 5,439,688, 5,418,219, 5,354,900, 5,298,246, 5,164,372, 4,900,549, 4,755,383, 4,639,435, 4,457,917, and 4,064,236. The combination used by the present invention may be preferably mixed with an excipient, carrier and/or diluent, and optionally, a preservative or the like pharmacologically acceptable vehicles as known in the art, see e.g., the above US patents. Examples of excipients include glucose, mannitol, inositol, sucrose, lactose, fructose, starch, corn starch, microcrystaline cellulose, hydroxypropylcellulose, hydroxypropylmethyl-cellulose, polyvinylpyrrolidone and the like. Optionally, a thickener may be added, such as a natural gum, a cellulose derivative, an acrylic or vinyl polymer, or the like.

The pharmaceutical composition is preferably provided in liquid, solid or semi-solid form. The liquid preparation is provided preferably as an oil suspension or microcapsule composition. A semi-solid composition is provided preferably as hydrous or oily gel or cream.

Tablets, hard tablets, capsules, and particularly soft gelatin capsules containing the combination or composition of the invention may be preferred, either as dietary supplements or as pharmaceutical dosage forms. In essence, any pharmaceutical dosage form suitable for oral administration may be used for delivering the combination of the invention.

The dosage of the PSE+DAG combination of the invention may depend upon the condition to be treated, the patient's age, sex and bodyweight, and will be determined by the attending physician or dietician. A preferred dosage for an adult may be from about 1.5 to about 6 g of PSE+DAG in oil dispersion (in accordance with the invention) per day, preferably 2-5 g, which shall comprise approximately 1300 mg of PSEs and up to 800 mg of DAGs.

Other formulations which may be in unit dosage forms may be as follows:

A combination of PSE and DAG dissolved in canola oil where the fatty acids of the sterol esters and the DAG resemble the fatty acid profile of canola oil, comprising including 31.5% wt PSE and 10% wt DAG. Each 4.5 g of this combination will contain at least 1300-1400 mg PSE (RDA) and 450 mg of DAG.

A combination of PSE and DAG dissolved in soybean oil, where the fatty acids of the sterol esters and the DAG resemble the fatty acid profile of soybean oil, comprising 31% wt PSE and 15.9% wt DAG. Each 4.5 g of this formulation will contain at least 1300-1400 mg PSE (RDA) and 700 mg of DAG.

A combination of PSE and DAG dissolved in canola oil comprising 61% wt PSE and 10% wt DAG. Each 2 g will contain at least 1300-1400 mg PSE (RDA) and 200 mg of DAG.

A combination of PSE and DAG dissolved in fish oil where the fatty acids of the sterol esters and the DAG resemble the fatty acid profile of fish oil, comprising 61% wt PSE and 8.3% wt DAG. Each 2 g will contain at least 1300-1400 mg PSE (RDA) and 150 mg of DAG.

A combination of PSE and DAG dissolved in canola oil where the fatty acids of the sterol esters and the DAG resemble the fatty acid profile of canola oil, comprising 23% wt PSE and 8% wt DAG. Each 6 g will contain at least 1300-1400 mg PSE (RDA) and 450 mg of DAG. Other PSE and DAG formulations as well as dilutions of all formulations are within the scope of the invention.

The present invention is particularly directed at a method of treating and/or preventing conditions related to any one of high cholesterol and triglycerides blood levels, serum oxidative stress, ox-LDL uptake by macrophages, macrophage oxidative status, foam cells formation and lipid-induced oxidative stress, said method consisting of orally administering a therapeutically effective dosage of the combination or composition of the invention, in the form of a food supplement, nutraceutical or pharmaceutical composition to a subject in need. Consequently, the method is also effective for the treatment of cardiovascular disorders, coronary heart disease, atherosclerosis, as well as cardiovascular disorders induced or manifested by other diseases such as diabetes mellitus, particularly Type II diabetes.

Alternatively, such conditions are to be treated by consumption of the dietary products in accordance with the invention.

Lastly, the present invention presents a method for improving health, consisting of administering a therapeutically effective dosage of the PSE+DAG combination or composition of the invention, as a dietary supplement, nutraceutical or pharmaceutical composition to a subject in need.

The present invention is defined by the claims, the contents of which are to be read as included within the disclosure of the specification.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

Materials:

Olive oil: commercial extra virgin olive oil, manufactured by Meshek Eger (Yokeneam HaMoshava, Israel).

Canola oil: commercial canola oil, manufactured by Shemen Taasiot (Haifa, Israel).

Fish oil: commercial fish oil, manufactured by Pronova (Lysaker, Norway).

TABLE 1
Composition of the oils used in the invention

| | | PSE + DAG | | Olive oil |
|---|---|---|---|---|
| | PSE + | in Olive oil | | enriched |
| | DAG in | (Enzymotec | PSE + DAG | with |
| Oil components % | Canola | FG | in Fish | phytosterol |
| (w/w) | oil | S7/1.75) | oil | esters |
| Phytosterol esters | 26 | 28.5 | 22.12 | 18 |
| Monoglycerides | 2.1 | 1.48 | 4.72 | 0.31 |
| Diglycerides | 14.9 | 14.62 | 20.02 | 0.81 |
| Triglycerides | 46.9 | 48.9 | 40.3 | |
| Free sterols | 3.1 | 1.5 | 5.2 | 0.2 |
| FFA | 7 | 5 | 5 | 3 |
| Glycerol | N.D. | N.D. | 2.6 | N.D. |
| Brassicasterol* | 0.54 | 0.46 | 0.82 | N.D. |
| Campasterol* | 5.43 | 4.58 | 4.93 | 0.009 |
| Stigmasterol* | 2.84 | 3.86 | 3.25 | 0.00142 |
| Betasitosterol* | 8.9 | 8.41 | 8.1 | 0.166 |

*levels of the different sterols, which can be either esterified or non-esterified

All materials were protected from light and odorous products at a temperature not exceeding 25° C.

Methods

Animal Studies

Free Radical Scavenging Capacity

The free radical-scavenging capacity of olive oil, olive oil+phytosterols, and PSE+DAG in olive oil was analyzed by the DPPH assay. DPPH (1,1-diphenyl-2-picryl-hydrazyl) is a radical-generating substance that is widely used to monitor the free radical scavenging abilities (the ability of a compound to donate an electron) of various anti-oxidants [Belinky, P. A. et al. (1998) Free Radic. Biol. Med. 24: 1419-291. The DPPH radical has a deep violet color due to its impaired electron, and radical scavenging can be followed spectrophotometrically by the loss of absorbance at 517 nm, as the pale yellow non-radical form is produced. 15 μl from stock solution of each sample were mixed with 1 mL of 0.1 mmol DPPH/L in ethanol and the change in optical density at 517 nm was continuously monitored.

Isolation of Mouse Peritoneal Macrophages

Mouse peritoneal macrophages (MPM) were harvested from the peritoneal fluid of the E^{o} mice (15-25 g) 4 days after intraperitoneal injection of thioglycolate (24 g/L) in saline (3 mL). Cells (10-20×10⁶/mouse) were washed 3 times with PBS and re-suspended to 10⁶/mL in DMEM containing 5% fetal calf serum (heat-inactivated at 56° C. for 30 min), 100 U penicillin/mL, 100 μg streptomycin/mL, and 2 mM glutamine. The cell suspension was plated into culture dishes and incubated in a humidified incubator (5% CO₂, 95% air) for 2 hours. The dishes were washed once with DMEM to remove non-adherent cells, and monolayers were incubated under similar conditions for 18 hours. Mouse peritoneal macrophages were isolated from 6 mice from each group, pooled and analyzed in duplicate or triplicate for each assay.

Macrophage Superoxide Release

The production of superoxide anion (O₂_{^{−}}) by mouse peritoneal macrophages was measured as the superoxide dismutase-inhibitable reduction of cytochrome C [Yanagitani Y. et al. (1999) Hypertension 33:335-91. Cells (1×10⁶/well) were incubated in 1 mL of HBSS containing acetyl cytochrome C (80 μmol/L). Superoxide production by the cells was stimulated by the addition of phorbol myristate acetate (PMA; 0.5μg/mL) for 1 hour. To some control samples, superoxide dismutase (SOD, 30 mg/L) was added. The amount of superoxide release was determined in the medium and was expressed as nmoles of superoxides/mg cell protein, using an extinction coefficient of E₅₅₀=21 mmol/L⁻¹cm⁻¹.

Macrophage Peroxide Content

Cellular peroxide levels were determined by flow cytometry using Dichlorofluorescin-diacetate (DCFH-DA) [Goupy, P. et al. (2003) Fr. Journal of Agricultural and Food Chemistry 51(3):615-622]. DCFH-DA is a non-polar dye that diffuses into the cells. In the cells it is hydrolyzed into the nonfluorescent derivate 2′,7′-DCFH, which is polar and trapped within the cells. Under oxidative stress, DCFH is oxidized to DCF (2′,7′-dichlorofluorescein), which is a fluorescent compound. Peritoneal macrophages (2×10⁶) were incubated with 2.5×10⁻⁵ mol/L DCFH-DA for 30 minutes at 37° C. Reaction was stopped by washes with PBS at 4° C. Cellular fluorescence was determined with a flow cytometry apparatus (FACS-SCAN, Becton Dickinson, San Jose, Calif., USA). Measurements were done at 510 to 540 nm after excitation of cells at 488 nm with an argon ion laser.

Serum Lipids Profile

Serum samples were analyzed for their lipid profile including total cholesterol and triglycerides, by using commercially available kits (Roche Diagnostics, Penzberg, Germany).

Serum Lipids Peroxidation

Serum was diluted 1:4 in PBS. Serum susceptibility to oxidation was determined by incubating serum sample with 100 mM of the free radical generating compound, 2′-2′-azobis 2′-amidinopropane hydrochloride (AAPH), which is an aqueous soluble azo compound that thermally decomposes to produce peroxyl radicals at a constant rate. The formation of thiobarbituric reactive substances (TBARS) and of lipid peroxides was measured and compared to serum that was incubated under similar conditions, but without AAPH.

PON1 Activity Measurements

PON 1 activity in serum was determined by measuring arylesterase activity, using phenylacetate as the substrate. Initial rates of hydrolysis were determined spectrophotometrically at 270 nm. The assay mixture included 1.0 mM phenylacetate and 0.9 mM CaCl₂ in 20 mM Tris HCl, pH 8.0. Non-enzymatic hydrolysis of phenylacetate was subtracted from the total rate of hydrolysis. The E270 for the reaction was 1,310 M⁻¹ cm⁻¹. One unit of arylesterase activity is equal to 1 μmol of phenylacetate hydrolyzed/min/ml. Purified enzyme has nearly 2000 units of arylesterase activity per mg protein.

Macrophage Oxidative Status

Cellular oxidative stress was examined in DCF-loaded macrophages by flow-cytometry using the conversion of non-fluorescent DCFH-DA to its fluorescent counterpart DCF as an index.

Macrophage-Mediated Oxidation of LDL

MPM were incubated with LDL (100 μg of protein/mL) for 18 hours, under oxidative stress (in the presence of 2 μmol/L of CuSO₄), after which the extent of LDL oxidation was determined by the TBARS assay.

Macrophage Uptake of Oxidized LDL

MPM were incubated with ¹²⁵I-labeled oxidized LDL (10 μg of protein/ml), and lipoprotein cell-association and degradation by these cells was determined. Lipoprotein cellular degradation was measured in the collected medium as the trichloroacetic acid (TCA)-soluble, non-lipid radioactivity, which was not due to free iodide. Lipoprotein degradation in a cell-free system was measured under identical conditions, and was subtracted from the total degradation. The remaining cells were washed three times with cold PBS and dissolved in 0.1 N NaOH for protein and cell-associated lipoproteins determination.

Statistical Analyses

Student t-test was used for statistical analysis of the results.

### Example 1

Antioxidative Effect of Olive Oil, Olive Oil+Phytosterols and PSE+DAG in Olive Oil Against Macrophage Lipid Peroxidation in E^{o} Mice

As mentioned above, oxidative stress is involved in the pathogenesis of atherosclerosis. Atherosclerosis is associated with lipids peroxidation of plasma LDL and in arterial cells, including macrophages [Aviram M. (2000) Free. Radic. Res. 33:S85-97]. Under oxidative stress, macrophage peroxide levels become increased, and macrophages generate reactive oxygen species, leading to their increased atherogenicity [Aviram M. (2000) id ibid].

The apolipoprotein E deficient (E⁰) mice are widely used as an animal model for atherosclerosis as they develop severe hypercholesterolemia and atherosclerotic lesions on a chow diet. Moreover, in E⁰ mice, accelerated atherosclerosis is associated with increased lipid peroxidation of plasma lipoproteins and arterial cells [Keidar S. (1998) Life Sci. 63:1-11].

Angiotensin II (Ang-11), a vasoconstrictor produced by the renin-angiotensin system, has been implicated in atherosclerosis. Ang-II activates macrophage NAD(P)H-oxidases, leading to increased macrophage lipid peroxidation [Rajagopalan S. et al. (1996) J. Clin. Invest. 97:1916-1923; Johnston R. B. Jr. (1984) Methods Enzymol. 105:365-9].

In the present Example, the anti-oxidative effect of three preparations of olive oil, designated olive oil+phytosterols, PSE+DAG in olive oil and olive oil, against macrophage oxidative stress was analyzed.

The following oil samples were tested (all diluted in water 1/2 vol./vol., stock solution):

1. Olive oil+phytosterols

2. PSE+DAG in olive oil

3. Olive oil

The antioxidative effect against macrophage oxidative stress of the PSE+DAG in olive oil in comparison to olive oil+phytosterols and olive oil alone, was analyzed by two parameters: (i) the ability to decrease macrophage peroxide content; and (ii) macrophage ability to release superoxide ions.

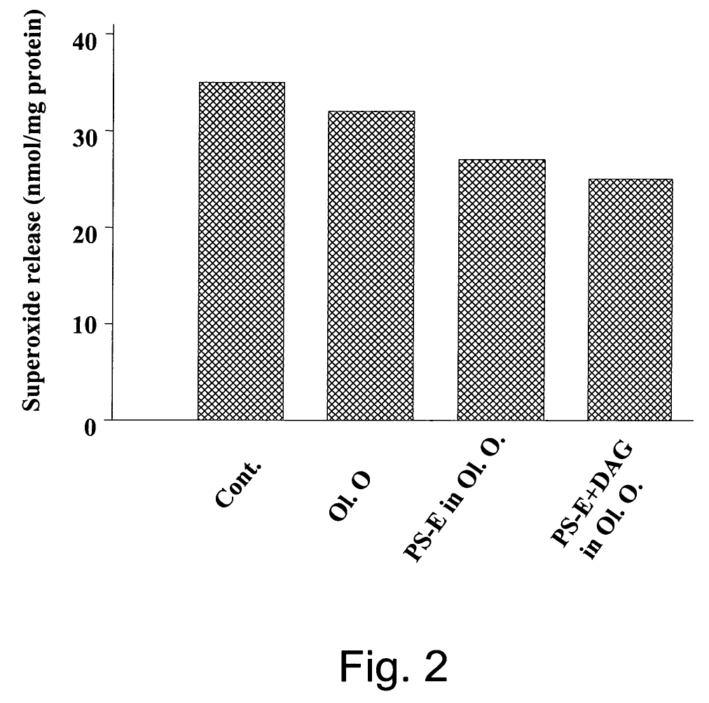

Mouse peritoneal macrophages were incubated with 50 □□l of stock solution/ml of either olive oil+phytosterols, PSE+DAG in olive oil and olive oil alone for 15 min, followed by a further incubation for 1 hour with Angiotensin II (10-7 M) to induce oxidative stress. Control cells were incubated with Angiotensin II alone. Macrophages were then analyzed for their peroxides content using the DCFH assay and for their ability to release superoxide ions (FIG. 2A, B).

1) Effect of Olive Oil, Olive Oil+Phytosterols, and PSE+DAG in Olive Oil on Macrophage Peroxide Content

Pre-incubation of the macrophages with both olive oil+phytosterols and PSE+DAG in olive oil but not with olive oil alone reduced the macrophage peroxide content compared to control macrophages incubated with Angiotensin II alone. The macrophage lipid peroxides content, using the DCFH assay, is assessed by two parameters; first, the mean fluorescence intensity emitted by DCF and second, the percentage of cells that are positive for fluorescence emission. Preincubation of macrophages with 50 μl/ml of olive oil+phytosterols or PSE+DAG in olive oil led to a reduction of 83% and 64% in macrophage mean fluorescence intensity compared to control cells, whereas olive oil at the same concentration had no effect on the macrophage mean fluorescence intensity compared to control cells (FIG. 2A). Similarly, preincubation of macrophages with 50 μl/ml of olive oil+Phytosterols or PSE+DAG in olive oil led to a reduction of 74% and 55% in percentage of positive cells for fluorescence compared to control cells, whereas olive oil at the same concentration had no effect on the percentage of positive cells for fluorescence compared to control cells (FIG. 2B).

2) Effect of Olive Oil, Olive Oil+Phytosterols and PSE+DAG in Olive Oil on Macrophages Superoxides Ions Release

Mouse peritoneal macrophages isolated from E⁰ mice were pre-incubated with 50 μl/ml of either PSE+DAG in olive oil, olive oil+phytosterols or olive oil alone for 15 minutes followed by a further incubation for 1 hour with Angiotensin II (10-7 M) to induce oxidative stress. Control cells were incubated with Angiotensin II alone.

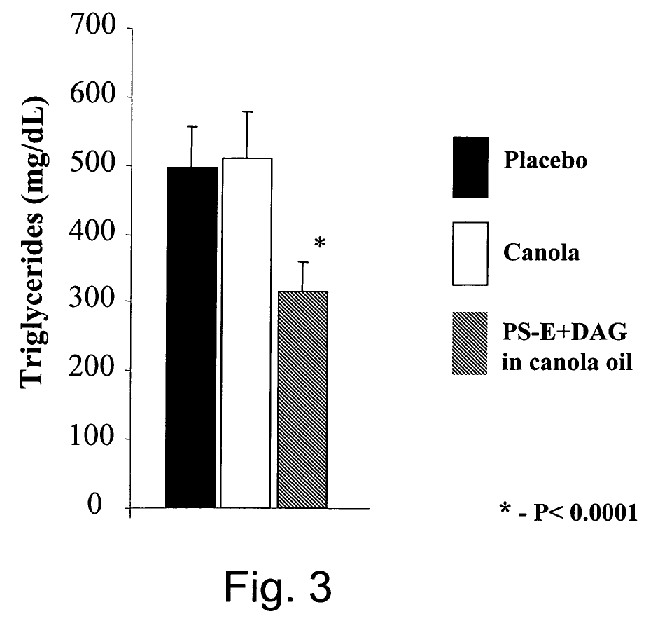

All three olive oils preparations analyzed in the present study inhibited to some extent, macrophage superoxide release induced by Angiotensin II. However, PSE+DAG in olive oil and olive oil+phytosterols were significantly more potent than olive oil alone. Pre-incubation of macrophages with 50 μl/ml of PSE+DAG in olive oil, olive oil+phytosterols or olive oil alone led to a reduction of 29%, 23% and only 9% respectively in macrophage superoxides anions release, compared to control cells incubated with Angiotensin II alone (FIG. 3).

Olive oil preparations enriched with phytosterols, in particular PSE+DAG in olive oil, exhibited significant anti-oxidative properties against macrophage lipid peroxidation. In contrast, whereas olive oil alone did not exhibit any effect. Most importantly, the PSE+DAG in olive oil composition was more potent than the olive oil+phytosterols preparation in its ability to reduce macrophage peroxide content and macrophage superoxide release.

These results suggest that olive oil and the additional components (phytosterols and diglycerides) can bind and internalize into the macrophages. In addition, olive oil enrichment with phytosterols enables the preparation of the invention to inhibit cellular oxidative systems (such as the NADPH oxidase and/or lypoxygenases) or to activate cellular anti-oxidant systems (such as the glutathione or superoxide dismutase systems). Furthermore, the addition of DAG to the olive oil+phytosterol preparation (resulting in the PSE+DAG in olive oil, in accordance with the invention) led to an additional antioxidative effect towards macrophage lipid peroxidation. The inventors thus speculate that DAG, which participates in numerous intracellular signal transduction pathways, could further affect the above cellular oxidative/antioxidative systems which are involved in Angiotensin II-mediated cellular oxidative stress, expressed as macrophage lipid peroxidation and superoxide release.

### Example 2

The effect of PSE+DAG in canola oil and PSE+DAG in fish oil on the atherogenicity of lipoproteins and macrophages, and on atherosclerosis development in the atherosclerotic apolipoprotein E deficient (E^{o}) mice model was investigated. Apolipoprotein E deficient (apoE^{o}) mice at 8 weeks of age were assigned randomly to the following groups (5 mice each) as described below. The mice received regular chow diet, and in addition, they were fed (via gavage) the following, once every three days:

Group I:

1. Placebo group: did not receive any addition of oil.

2. Canola oil group (control): were fed with 60 μl of canola oil.

3. PSE+DAG in canola oil group: were fed with 60 μl of PSE+DAG in canola oil.

Group II:

1. Placebo group: did not receive any addition of oil.

2. PSE+DAG in fish oil: were fed with 60 μl of PSE+DAG in fish oil.

Each mouse consumed approximately 5 mL of water/day, and 5 g of chow/day.

Oil Preparation for Feeding

The amounts of PSE+DAG in canola oil and PSE+DAG in fish oil fed to the mice were based on the following:

The recommended phytosterols dosage for humans is 1.5 gr of phytosterols/day. Based on 18.1% phytosterols in each sample, the dosage for PSE+DAG in canola oil and PSE+DAG in fish oil for humans is therefore 1.5/0.18=8.33 gr/day/person. For mice, the body weight should be taken into consideration (60,000 gr human body weight/20 gr mouse body weight=3000), thus the daily dosage for mouse is 8.33 gr/3000=2.78 mg/day/mouse, which is equal to 2.78/0.93=2.99 mL/day/mouse. Since the experiment is done for a limited period, the dosage used was 5-fold higher. Thus, each mouse was administered 15 mL of oil/day (60 mL/4 days/mouse).

At the end of the experimental period, blood samples were collected from all mice for serum separation and analyses. Within each experimental group, the blood sample of each mouse was analyzed individually. The following parameters were analyzed in the serum:
1. Determination of lipids, including total cholesterol and triglycerides levels.
2. Determination of serum oxidative status.
3. Determination of paraoxonase, measured as arylesterase activity.

MPM were harvested prior to removal of the heart and aorta. The mice were anesthetized with ethyl ether in a local nasal container.

The experimental protocol (No. IL-066-10-2001) was approved by the Animal Care and Use Committee of the Technion Israel Institute of Technology (Haifa, Israel).

FIG. 3 shows that the consumption of PSE+DAG in canola oil resulted in a remarkable and significant reduction in the levels of triglycerides in the serum (36%), in comparison with placebo (p<0.001).

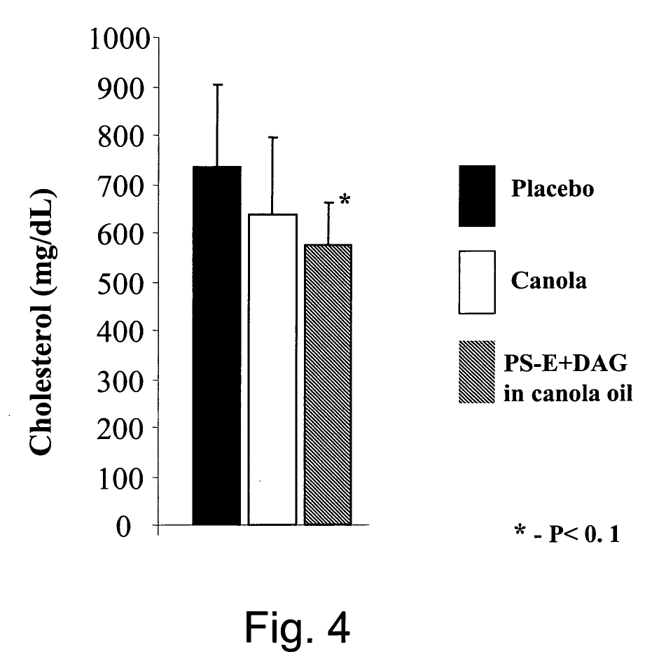

Similarly, FIG. 4 shows that particularly PSE+DAG in canola oil demonstrated a tendency to reduce total cholesterol levels in the serum (P<0.1).

FIG. 5 shows that PSE+DAG in canola oil treatment resulted in a drastic and highly significant (p<0.001) reduction of the serum susceptibility to AAPH-induced oxidation by 63% (in comparison to placebo). PSE+DAG in fish oil demonstrated a similar tendency, reducing lipid peroxides by 16% in comparison with placebo. The different efficacy between these two matrices could be attributed, at least in part, to the well documented tendency of these polyunsaturated fatty acids towards generation of lipid peroxidation products.

FIG. 6 shows an interesting result. Whereas canola oil consumption induced a significant reduction in the levels of serum PON1 activity (p<0.1), which may be detrimental for atherosclerosis [Mackness, B. et al. [2003] Circulation 107:2775-9], consumption of PSE+DAG in canola oil or in fish oil restored PON1 activity, to levels comparable of that of untreated (Placebo group) mice. Thus, consumption of PSE+DAG in canola oil and PSE+DAG in fish oil is beneficial for maintaining effective levels of PON1 activity.

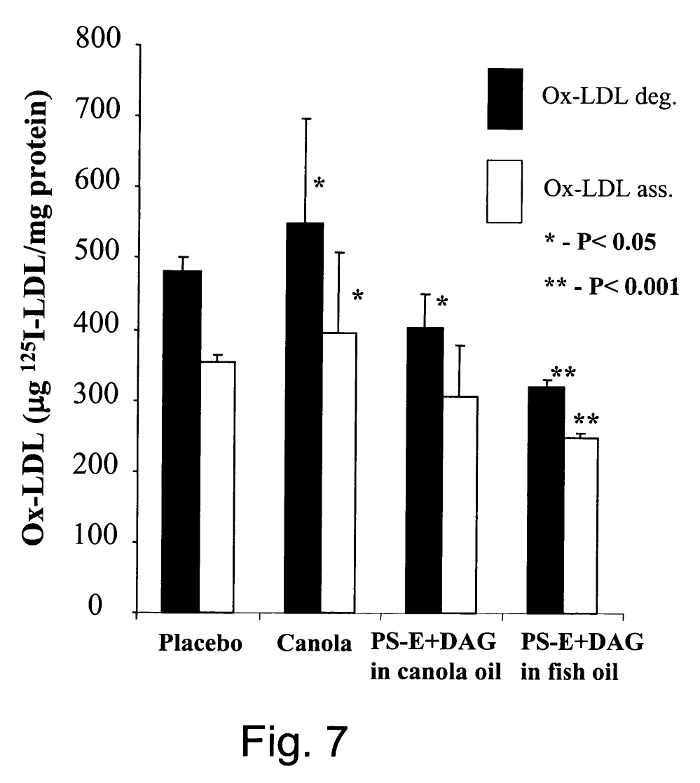

FIG. 7 demonstrates that consumption of PSE+DAG in canola oil caused reduction of ox-LDL association (16%) and degradation (14%) (p<0.05), resulting in increased MPM abilities to sustain ox-LDL, which can be correlated to a decreased oxidation status, and to a larger extent, PSE+DAG in fish oil displayed a similar effect, also causing reduction of ox-LDL association (34%) and degradation (30%) (p<0.001). In contrast, canola oil consumption resulted in a slight increased (p value<0.05) of both ox-LDL association and degradation (4% and 11%, respectively, in comparison to placebo).

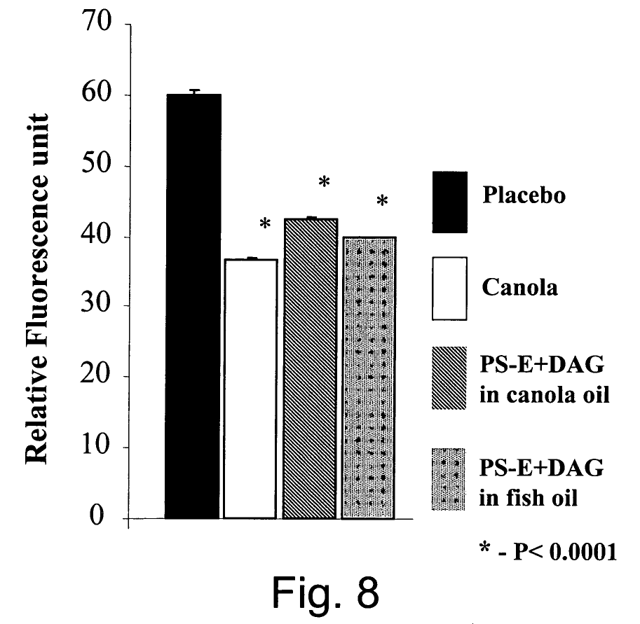

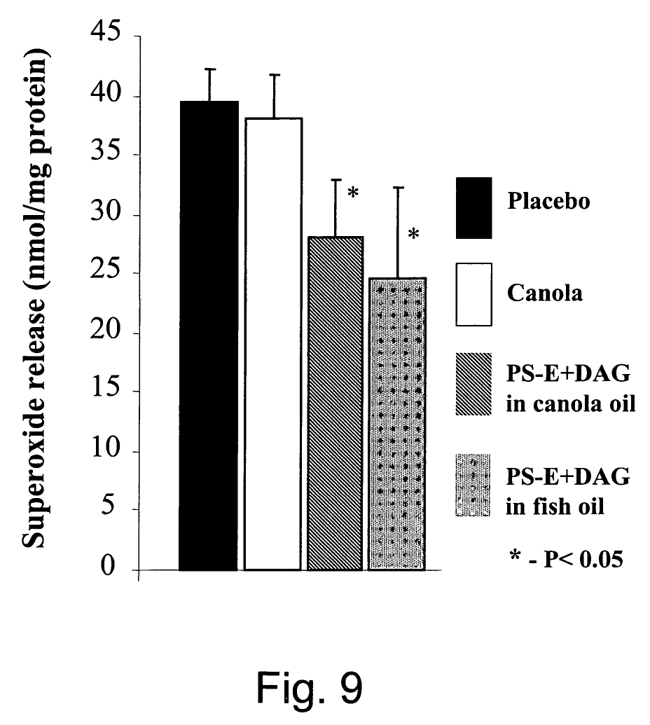

FIG. 8 shows that consumption of either PSE+DAG in canola oil or PSE+DAG in fish oil significantly reduced the oxidative status of E⁰ mice macrophages (p<0.0001). PSE+DAG in fish oil reduced macrophages oxidative status by 34% in comparison to placebo, while PSE+DAG in canola oil reduced it by 29% in comparison to placebo. Thus, both PSE+DAG in fish oil and PSE+DAG in canola oil are effective in reducing the oxidative status of macrophages. Consistent with these results, in FIG. 9, the inventors show that, similarly, consumption of either PSE+DAG in fish oil or PSE+DAG in canola oil also significantly reduced the PMA-induced release of superoxide anions in macrophages (p<0.05).

### Example 3

### Human Study

Methods

Human Study Design

This study was designed as a randomized, single-blind, cross-over clinical intervention trial. This experiment involved testing the effects of dietary matrices containing specific fatty acid and/or plant sterol mixtures included in normal diets, compared to non-supplemented diets, on circulating lipid levels, in moderately overweight subjects with elevated serum lipid levels for whom dietary modification is the primary and at times only therapeutic recommendation.

Patients

Twenty-four (24) volunteers (11 male, 13 female, age 30-65 yr) with LDL-cholesterol concentrations >130 mg/dL were recruited for this study. Body mass indices (BMI) of subjects ranged from 23-32 kg/M², except for three subjects in which BMI was between 21.4 and 23 kg/M². Twenty-one volunteers completed the study (11 male, 10 female).

Diets

All volunteers underwent a feeding trial according to a semi-randomized Latin square cross-over design containing three dietary phases, each four week in duration. The control diet was provided first, then the other phases randomized. Each feeding period was separated by a 4-week washout interval, during which volunteers consumed their typical diets without restriction. The composition of the diets was similar with respect to the food and nutrient content. The basic diet contained 30% of energy as fat (see control diet), 80 mg cholesterol/ 1000 kcal, 12 g fiber/ 1000 kcal, 15% energy as protein and 55% as carbohydrates. The variable component was the treatment oil. Diets were designated as:
Control group: baseline plant sterol level of about 200 mg/day, where the dietary fat was comprised at 70% energy as oleic acid-enriched vegetable oil.
PSE+DAG group: 1.7 g/day soy sterols esterified to olive oil fatty acids (predominantly oleic acid), contained in a diglyceride (DAG) and triglyceride containing olive oil (total amount=9 g/d).

All meals were equicaloric and breakfast was consumed each day under supervision. The treatment oil was given within the breakfasts consumed every day at the clinic under supervision. Volunteers were instructed to eat and drink only materials given to them by the Clinical Nutrition Research Unit (McGill University, Montreal, Canada), except for water. Study volunteers were encouraged to maintain their usual level of physical activity. Energy requirements were estimated using the Mifflin equation and multiplied by an activity factor of 1.7%. Energy intakes were adjusted over the initial 2-week period to maintain constant body weight and kept constant for the remaining 4 weeks and the other treatment phases.

Blood Lipid Analyses

On days 1, 2, 28 and 29, blood samples were obtained in the fasting state. On day 28, subjects reported at the clinic 4 hours after they consumed their normal experimental breakfast so that a blood sample was obtained in the postprandial state. Plasma was immediately separated and stored at -80° C. until analysis. The general lipid profile (Total, HDL and LDL cholesterol as well as triglycerides) was measured in samples taken on days 1, 2, 28 and 29 of each phase. Plasma total cholesterol and triglyceride in plasma, and HDL sub-fractions were determined by automated methods in duplicate on an Abbott Spectrum CCX Analyzer (Abbott, Dallas, Tex.) utilizing enzymatic reagents (Abbott A-GENT). LDL cholesterol was calculated by the Friedewald equation. If triglycerides were higher than 400 mg/dL, then LDL cholesterol levels were directly measured in plasma samples (N-geneous LDL-C assay, Equal Diagnostics), using a method in which LDL precipitated by the dextran/magnesium sulfate method in order to separate them from HDL. Apolipoproteins A and B100 were measured by nephelometry in samples from days 1, 2, 28 and 29. Lipoprotein (a) was measured by nephelometry in samples from days 1 and 28.

Plasma thiobarbituric acid reactive substances (TBARS) content, as a marker of its susceptibility to oxidative stress was determined using a commercial kit (TBARS assay kit, OXI-tek).

Results

Change in Total Cholesterol Concentrations

Supplementation of mildly overweight hypercholesterolemic subjects' diet with PSE+DAG demonstrated a distinct impact on total plasma cholesterol concentrations (P-value<0.0001; see FIG. 10). Compared to control MUFA based OA-enriched oil diet, PSE+DAG consumption induced a significantly higher hypocholesterolemia effect (−4% vs. −12%, respectively; P=0.03). In terms of absolute cholesterol concentrations reduction, the volunteers that consumed these diets reduced 13 mg/dL and 30 mg/dL; from initial average level of 240 mg/dL and 243 mg/dL to a final average levels of 228 mg/dL and 213 mg/dL, respectively. It must also be noted that this background diet, contained limited amount of cholesterol (not more than 80 mg/1000 kcal), which could also play part in the total cholesterol levels reduction effect. With respect to the American Heart Association (AHA) recommendations towards blood lipids levels [Expert Panel on Detection, Evaluation, and Treatment of High Blood Cholesterol in Adults (2001) J.A.M.A. 285:2486-2497], suggesting the desirable total cholesterol levels in healthy individuals to be below 200 mg/dL, the volunteers who consumed the vegetable-oil diet reduced 31% of the total cholesterol levels required to meet the target. However, following PS-E+DAG feeding the serum total cholesterol levels were markedly reduced towards these optimal level inasmuch as 71%.

Change in LDL-Cholesterol Concentrations

As shown in FIG. 11, the hypocholesterolemic effect of PS-E+DAG was more prominent, as expected, in the reduction of LDL cholesterol plasma levels (from baseline levels of 154±8 mg/dL to endpoint 135±7 mg/dL; P-value=0.0004; while control vegetable oil consumption resulted in a significant effect on reduction of LDL cholesterol concentrations; however to a somewhat lower extent (from baseline levels of 156±8 mg/dL to endpoint 148±7 mg/dL; P-value=0.051; see FIG. 11). Indeed, in a paired analysis of the percentages of change in the LDL-cholesterol concentrations after supplementation with the PSE+DAG mixture compared to those observed after control oil supplementation, a marked tendency was demonstrated (−11.91% versus −4.13%, respectively; P=0.058). These hypercholesterolemic overweight volunteers, fed for four weeks with control MUFA based OA-enriched oil diet or PSE+DAG followed by four weeks of washout and counter supplementation, reduced their blood LDL-c levels by 31% or 81%, respectively, with respect to AHA definition of LDL-C levels of 130 mg/dL as near to optimal levels.

Change in ApoB Concentrations

Lipoproteins and their associated apolipoproteins, like LDL-c and apo B, reduction was reported following OA-enriched olive oil diet, as compared to high omega-6 enriched diet in diabetic type 2 patients [Madigan C., et al., (2000) Diabetes care 23:1472-1477]. Therefore, a significant reduction in these volunteers’ apo B levels following base oil consumption was expected (from baseline values of 1.19±0.07 g/L to endpoint 1.13±0.05 g/L; P-value=0.016; FIG. 12). As previously shown [Amundsen A L., et al. (2002) Am. J. Clin. Nutr. 76:338-344] for plant sterol esters spreads, a more pronounced effect was demonstrated following PS-E+DAG supplementation to these hypercholesterolemic volunteers (from baseline values of 1.21±0.06 g/L to endpoint 1.09±0.06 g/L; P-value=0.0002; FIG. 12). Moreover, a comparison of the reducing effect of PSE+DAG on endpoint apo B concentrations

(−9.85%) to the one obtained by the control diet (−2.77%), suggested a statistically significant difference between these dietary treatments effects (P=0.022). Interestingly, Kondo and colleagues [Kondo A., et al. (2002) J Atheroscler Thromb. 9:280-287] suggested a strong correlation between oxidized-LDL and apo B concentrations, having a turning point at apo B levels above 1.15 g/L. Both diets, were shown to be beneficial in obtaining this anti-oxidant outcome, albeit PSE+DAG presenting a more potent effect.

Change in ApoB/ApoA Ratio

Apo B, apo A-I and the apo B/apo A-I ratio have been reported as better predictors of cardiovascular events than LDL-C and they even retain their predictive power in patients receiving lipid-modifying therapy [Walldius J. and Jungner I. (2004) J. Intrn. Med. 255:188-205]. As may be seen from FIG. 13, in addition to the aforementioned reduction in apo B levels, introducing PSE+DAG to the base MUFA-enriched diet resulted in marked and significantly reduced levels of apoB/apoA ratios (from baseline values of 0.89±0.05 to endpoint 0.84±0.05; P-value=0.047; while no comparable effect was obtained following control diet (from baseline values of 0.91±0.04 to endpoint 0.89±0.04; P-value=0.19; FIG. 13). No significant differences were observed between the effects of these treatments on apoB/apoA ratios in % change (−4.01% and −1.35%, respectively; P=0.22). Similar observations were reported following plant sterol or stanol esters spreads consumption [Amundsen A L., et al. (2002) id ibid; Hallikainen M A. et al. (2000) J. Nutr. 130:767-776].

Change in Lipoprotein (a) Levels

Lipoprotein(a) (Lp(a)) is an LDL-like particle to which apo A is attached through a disulfide bound to apo B. Increased plasma level of Lp(a) is an independent predictor of the presence of angiographically documented and clinical CAD, particularly in patients with hypercholesterolemia [Danesh J., et al. (2000) Circulation 102:1082-1085]. Recently [Tsimikas S., et al. (2005) N. Engl. J. Med. 353:46-57], it was suggested that the atherogenicity of Lp(a) may be mediated in part by associated proinflammatory oxidized phospholipids in human plasma. The authors were able to demonstrate an association between the oxidized phospholipid:apo ratio and extent of angiographically documented CAD, which was independent of all lipid-related risk factor but Lp(a). MUFA like oleic acid, which was the principal fat in all diets, has been shown in a previous study to increase Lp(a) levels in comparison to saturated fats [Vessby B. et al. (2001) Diabetologia 44:312-319]. It is therefore conceivable that the tested unsaturated fatty acid containing vegetable oil diet may favor an increase in Lp(a) concentrations regardless of the supplement given. Indeed, hypercholesterolemic volunteers fed with control diet presented significantly elevated levels of Lp(a) (from baseline values of 0.18±0.03 g/L to endpoint 0.22±0.04 g/L; P-value=0.0003; FIG. 14). However, esterification of monounsaturated fatty acids to plant sterols in a DAG containing matrix (PS-E+DAG) maintained Lp(a) concentrations (from baseline values of 0.19±0.03 g/L to endpoint 0.19±0.03 g/L; P-value=0.41; FIG. 14). Interestingly, a one-year study of conjugated linoleic acid (CLA) administration to healthy overweight adults [Gaullier J. M., et al., (2004) Am. J. Clin. Nutr. 79:1118-1125], demonstrating beneficial effect on body fat mass, also indicated deleterious effect on lipoproteins levels including inducing elevation of Lp(a) levels, following either the free fatty acid or the triglycerides CLA format.

Change in Triglycerides Levels

Recently, an elevated triglycerides concentration was suggested to be a univariate predictor of CVD. Specifically, 88 mg/dL increase in triglycerides levels was associated with a 14% increase in CVD risk in men and a 37% increase in women [Austin M A., et al. (1998) Am. J. Cardiol. 81:7B-12B]. Olive oil based diet was already demonstrated to induce a marked (10%) triglycerides levels reduction, comparing with an average American diet [Kris-Etherton P M., et al., (1999) Am. J. Clin. Nutr. 70:1009-1015]. Therefore, the reduction in the fasting triglycerides levels of the different diets was anticipated, as the base diet oil was OA-enriched (control diet from 191±32 mg/dL to 165±25 mg/dL, P-value=0.038 and PSE+DAG in olive oil 182±27 mg/dL to 155±22 mg/dL, P-value=0.031). The extent of the PSE+DAG effect on fasting triglycerides levels (−9.9%) was at least somewhat higher than what was obtained for control high oleic diet (−5.3%), which could also be appreciated from the difference in the median values of % of change (−10.4% and −3.7%, respectively). It should be also noted that the tested subjects were normolipidemic but presented elevated levels of LDL-C. It is well established [Normen L., et al. (2004) Curr Med Chem Cardiovasc Hematol Agents 2:1-12] that even potent dietary hypotriglyceridemic agents like fish oil demonstrate different effect in normolipidemic as opposed to hyperlipidemic subjects.

Change in Oxidative Stress Levels

Aviram and Eias [Aviram M. and Eias K. (1993) Ann. Nutr. Metab. 37:75-841 had demonstrated that in vitro incubation of LDL with copper ion in the presence of linoleic acid (LA) resulted with a 22% elevation of LDL oxidation, as opposed to marked dose-dependent inhibition of lipoprotein oxidation by pre-incubation with OA. Moreover, a recent study [Madigan C., et al., (2000) Diabetes care 23:1472-1477] suggested that, in type 2 diabetes, an OA-rich Mediterranean-type diet versus a LA-enriched diet may reduce the risk of atherosclerosis by decreasing the number of chylomicron remnant particles. In consistence, control diet, enriched with MUFA, demonstrated a mild tendency toward reducing the oxidative levels, as recorded using TBARS analysis (from baseline values of 1.11±0.48 mmol/L to endpoint 0.91±0.11 mmol/L; P-value=0.10; FIG. 15). Importantly, following PSE+DAG consumption there was a more pronounced and significant reduction in plasma oxidative stress levels (from baseline values of 1.01±0.21 mmol/L to endpoint 0.83±0.15 mmol/L; P-value=0.005; FIG. 15). These observations could be attributed in part to the aforementioned obtained indications for marked reduction of total cholesterol, LDL-C, apo B, and triglycerides while maintaining Lp(a) levels, which were previously shown to be correlated with elevated serum anti-oxidant capacity levels.

1. A method for reducing blood levels of both cholesterol and triglycerides and/or for lowering serum, serum LDL and macrophage oxidation levels, inhibiting the formation of foam cells and/or preventing the deleterious effects generated by lipid-induced oxidative stress in a subject in need, comprising administering to said subject a dietary nutrient or food supplement comprising an edible combination of diacylglycerol(s) (DAG), mainly 1,3-diacylglycerol(s), and phytosterol and/or phytostanol ester(s) (PSE), wherein the ratio of PSE:DAG in said combination is at least 1.

2. The method of claim 1, wherein said combination of DAG and PSE is dissolved or dispersed in an edible oil and/or fat.

3. The method of claim 2, wherein said oil is a natural and/or edible oil, preferably olive oil, soybean oil, sunflower oil, safflower oil, canola oil, palm oil, avocado oil, sesame oil and fish oil, more preferably olive oil, canola oil or fish oil.

4. The method of claim 2, wherein said fat is any natural fat, preferably butter fat, anhydrous milk fat, cocoa butter or lard.

5. The method of claim 1, wherein the fatty acid residues of the DAG and of the PSE optionally correspond to the fatty acid residues of the oil from which it is derived, such as oleic, palmitic, palmitoleic, stearic, linoleic, linolenic, and eicosanoic acid residues.

6. The method of claim 1, wherein the phytosterol ester(s) is/are fatty acid ester(s) of stigmasterol, sitosterol, beta-sitosterol, brassicasterol, campesterol and/or 5-avenasterol and isomers and derivatives thereof and said phytostanol ester(s) is/are fatty acid ester(s) of beta-sitostanol, campestanol and/or stigmastanol and isomers and derivatives thereof.

7. The method of claim 1, wherein said combination further comprises conventional ingredients of nutritional compositions.

8. The method of claim 1, wherein the weight ratio between phytosterol and/or phytostanol esters and diacylglycerol(s) and phytosterol and/or phytostanol ester(s) in said combination is from about 15:1 to 1:1, preferably about 10:1 to about 1:1, more preferably 5:1 to 1:1 and particularly about 2:1.

9. The method of claim 1, wherein the amount of diacylglycerol(s) in said combination is at least 1 wt %.

10. The method of claim 1, wherein the amount of phytosterol and/or phytostanol ester(s) in said combination is at least 1 wt %.

11. The method of claim 1, wherein the amount of diacylglycerol(s) in said combination is from about 1 to about 99 wt %, preferably from about 4 to about 70 wt %, particularly from about 7 to about 48 wt %, more particularly from about 10 to about 22 wt % and the amount of phytosterol and/or phytostanol ester(s) in said combination is from about 1 to about 99 wt %, preferably from about 5 to about 70 wt %, more particularly from about 7 to about 60 wt %, specifically from about 10 to about 60 wt %, more particularly from about 7 to about 35 wt % and more specifically from about 20 to about 35 wt %.

12. The method of claim 2, wherein said combination consists of 15 wt % DAG, mainly 1,3-DAG(s) and 25 wt % total PSE(s) dissolved or dispersed in any one of olive, canola and fish oil.

13. A method for treating and/or preventing cardiovascular disorders and diseases related thereto, particularly coronary heart disease, atherosclerosis and cardiovascular disorders induced or manifested by other diseases such as metabolic syndrome and any of the conditions involved therein, such as diabetes mellitus and more particularly Type II diabetes, in a subject in need, comprising administering to said subject a dietary nutrient or food supplement comprising an edible combination of diacylglycerol(s) (DAG), mainly 1,3-diacylglycerol(s), and phytosterol and/or phytostanol ester(s) (PSE), wherein the weight ratio of PSE:DAG in said combination is at least 1.

14. The method of claim 13, wherein said combination of DAG and PSE is dissolved or dispersed in an edible oil and/or fat.

15. A method for reducing blood levels of both cholesterol and triglycerides and/or for lowering serum, serum LDL and macrophage oxidation levels, inhibiting the formation of foam cells and/or preventing the deleterious effects generated by lipid-induced oxidative stress in a subject in need, comprising administering to a subject an orally administrable pharmaceutical composition comprising a combination of diacylglycerol(s) (DAG), mainly 1,3-diacylglycerol(s) and phytosterol and/or phytostanol ester(s) (PSE), wherein the weight ratio of PSE:DAG in said combination is at least 1, and further comprising pharmaceutically acceptable additives, diluents, excipients and/or carriers.

16. The method of claim 15, for the treatment and/or prevention of cardiovascular disorders and diseases related to the same, coronary heart disease and/or atherosclerosis and cardiovascular disorders induced or manifested by other diseases such as metabolic syndrome and any of the conditions involved therein, particularly diabetes mellitus, and more particularly Type II diabetes.

17. The method of claim 15, wherein said combination of DAG and PSE is dissolved or dispersed in an edible oil and/or fat.

18. The method of claim 17, wherein said oil is a natural and/or edible oil, preferably olive oil, soybean oil, sunflower oil, safflower oil, canola oil, palm oil, sesame oil, avocado oil and fish oil, preferably olive oil, canola oil or fish oil.

19. The method of claim 18, wherein the fat is any natural fat, preferably butter fat, cocoa butter, anhydrous milk fat and lard.

20. The method of claim 15, wherein the fatty acid residues of the DAG and the PSE correspond to the fatty acid residues of the oil from which it is derived, such as oleic, palmitic, palmitoleic, stearic, linoleic, linolenic, and eicosanoic acid residues.

21. The method of claim 15, wherein the phytosterol ester(s) is/are fatty acid ester(s) of stigmasterol, sitosterol, beta-sitosterol, brassicasterol, campesterol and/or 5-avenasterol and isomers and derivatives thereof and said phytostanol ester(s) is/are fatty acid ester(s) of beta-sitostanol, campestanol and/or stigmastanol and isomers and derivatives thereof.

22. The method of claim 15, wherein the weight ratio between phytosterol and/or phytostanol esters and diacylglycerol(s) and phytosterol and/or phytostanol ester(s) in said combination is from about 15:1 to 1:1, preferably about 10:1 to about 1:1, more preferably 5:1 to 1:1 and particularly about 2:1.

23. The method of claim 15, wherein the amount of diacylglycerol(s) in said combination is at least 1 wt %.

24. The method of claim 15, wherein the amount of phytosterol and/or phytostanol ester(s) in said combination is at least 1 wt %.

25. The method of claim 15, wherein the amount of diacylglycerol(s) in said combination is from about 1 to about 99 wt %, preferably from about 4 to about 70 wt %, particularly from about 7 to about 48 wt %, more particularly from about 10 to about 22 wt % and the amount of phytosterol and/or phytostanol ester(s) in said combination is from about 1 to about 99 wt %, preferably from about 5 to about 70 wt %, more particularly from about 7 to about 60 wt %, specifically from about 10 to about 60 wt %, more particularly from about 7 to about 35 wt % and more specifically from about 20 to about 35 wt %.

26. The method of claim 15, for the treatment and/or prevention of cardiovascular disorders and diseases related to the same, coronary heart disease and atherosclerosis and cardiovascular disorders induced or manifested by other diseases such as metabolic syndrome and any of the conditions involved therein, particularly diabetes mellitus, more particularly Type II diabetes.

27. The method of claim 15, for reduction and/or prevention of any one of elevated cholesterol and triglycerides blood levels, serum oxidative stress, oxidative LDL uptake by macrophages, macrophage oxidative status, foam cell formation, and deleterious effects generated by lipid-induced oxidative stress.

28. A method for reducing blood levels of cholesterol and triglycerides and/or reducing LDL oxidation in a subject in need, comprising administering to said subject a pharmaceutical composition consisting substantially of 15 wt % DAG(s), mainly 1,3-DAG(s) and 25 wt % total PSE(s) dissolved or dispersed in olive oil, wherein said composition is characterized by having the effect of reducing blood levels of both cholesterol and triglycerides and/or for lowering serum, serum LDL and macrophage oxidation levels, inhibiting the formation of foam cells and/or preventing the deleterious effects generated by lipid-induced oxidative stress.

29. A method for maintaining PON1 activity levels in a patient in need, thereby reducing and/or preventing LDL oxidation and attenuating CVD and/or susceptibility of diabetic patients to vascular complications, comprising administering to said patient a dietary nutrient or food supplement or a pharmaceutical composition comprising an edible combination of diacylglycerol(s) (DAG), mainly 1,3-diacylglycerol(s), and phytosterol and/or phytostanol ester(s) (PSE), wherein the ratio of PSE:DAG in said combination is at least 1.

30. A method for maintaining Lp(a) levels and reducing and/or preventing the extent of angiographically documented Coronary Artery Disease in a subject in need comprising administering to said patient a dietary nutrient or food supplement or a pharmaceutical composition comprising an edible combination of diacylglycerol(s) (DAG), mainly 1,3-diacylglycerol(s), and phytosterol and/or phytostanol ester(s) (PSE), wherein the ratio of PSE:DAG in said combination is at least 1.

31. A dietary nutrient or food supplement comprising an edible composition for reducing blood levels of both cholesterol and triglycerides and/or for lowering serum, serum LDL and macrophage oxidation levels, inhibiting the formation of foam cells and/or preventing the deleterious effects generated by lipid-induced oxidative stress, said composition comprising a combination of diacylglycerol(s) (DAG), mainly 1,3-diacylglycerol(s) and phytosterol and/or phytostanol ester(s) (PSE) wherein the ratio of PSE to DAG in said combination is at least 1, optionally further comprises conventional ingredients of nutritional compositions.

32. The dietary nutrient of claim 31, wherein said combination is dissolved or dispersed in an edible oil and/or fat.

33. The dietary nutrient or food supplement of claim 31, wherein said oil is a natural and/or edible oil, preferably olive oil, soybean oil, sunflower oil, safflower oil, canola oil, palm oil, avocado oil, sesame oil and fish oil, more preferably olive oil, canola oil or fish oil.

34. The dietary nutrient or food supplement of claim 33, wherein said fat is any natural fat, preferably butter fat, anhydrous milk fat, cocoa butter or lard.

35. The dietary nutrient or food supplement of claim 31, wherein the fatty acid residues of the DAG and of the PSE optionally correspond to the fatty acid residues of the oil from which it is derived, such as oleic, palmitic, palmitoleic, stearic, linoleic, linolenic, and eicosanoic acid residues.

36. A dietary nutrient or food supplement of claim 31, wherein the phytosterol ester(s) is/are fatty acid ester(s) of stigmasterol, sitosterol, beta-sitosterol, brassicasterol, campesterol and/or 5-avenasterol and isomers and derivatives thereof and said phytostanol ester(s) is/are fatty acid ester(s) of beta-sitostanol, campestanol and/or stigmastanol and isomers and derivatives thereof.

37. The dietary nutrient or food supplement of claim 31, the weight ratio between phytosterol and/or phytostanol esters and diacylglycerol(s) and phytosterol and/or phytostanol ester(s) in said combination is from about 15:1 to 1:1, preferably about 10:1 to about 1:1, more preferably 5:1 to 1:1 and particularly about 2:1.

38. The dietary nutrient or food supplement of claim 31, wherein the amount of diacylglycerol(s) in said composition is at least 1 wt %.

39. The dietary nutrient or food supplement of claim 31, wherein the amount of phytosterol and/or phytostanol ester(s) in said combination is at least 1 wt %.

40. The dietary nutrient or food supplement of claim 31, wherein the amount of diacylglycerol(s) in said combination is from about 1 to about 99 wt %, preferably from about 4 to about 70 wt %, particularly from about 7 to about 48 wt %, more particularly from about 10 to about 22 wt % and the amount of phytosterol and/or phytostanol ester(s) in said combination is from about 1 to about 99 wt %, preferably from about 5 to about 70 wt %, more particularly from about 7 to about 60 wt %, specifically from about 10 to about 60 wt %, more particularly from about 7 to about 35 wt % and more specifically from about 20 to about 35 wt %.

41. The dietary nutrient or food supplement of claim 31, wherein said composition consists of 15 wt % DAG, mainly 1,3-DAG(s) and 25 wt % total PSE(s) dissolved or dispersed in any one of olive, canola and fish oil.
